(19) United States
(12) Patent Application Publication
(10) Pub. No.: US 2010/0209512 A1
(43) Pub. Date: Aug. 19, 2010

(54) Particle size-structured parenteral dispersions

(75) Inventors: David F. Driscoll, Bridgewater, MA (US); David F. Nicoli, Goleta, CA (US)

Correspondence Address: BUCHANAN, INGERSOLL & ROONEY PC, POST OFFICE BOX 1404, ALEXANDRIA, VA, 22313-1404, US

(73) Assignee: Stable Solutions LLC, Goleta, CA (US)

(21) Appl. No.: 12/379,149

(22) Filed: Feb. 13, 2009

(51) Int. Cl. A61K 9/14

(52) U.S. Cl. 424/489

(57) ABSTRACT

A Drug/Adjuvant Delivery System (D/A DS), and associated method, are disclosed. An exemplary D/A DS system includes a liquid carrier; and a particle-size structured dispersion of solid and/or liquid particles suspended in the liquid carrier.

### BACKGROUND

1. Field

The present disclosure relates to parenteral emulsions, suspensions and dispersions (hereinafter referred to inclusively, without loss of generality intended or implied, by using the single term, “dispersions”) having a particle size distribution (PSD) or globule size distribution (GSD) that has been deliberately designed, or “structured”, in order to, for example, optimize the delivery of pharmaco-therapeutic or pharmaco-adjunctive agents contained in infusions for treatment of disease with respect to the efficacy of their deposition at the intended target site, and/or the intended occlusion of surrounding blood vessels. The terms “particles” and “droplets”, or “globules”, are used interchangeably throughout this disclosure with respect to the “structuring” of dispersions comprised therefrom. As above, there is no loss of generality intended or implied by the use of one of these terms as opposed to another, and the term “particle” is used generally to describe the suspended phase(s) of the dispersion. Also, there are specific kinds of known particles, called vesicles and liposomes, that are used as vehicles for drug delivery, and these kinds of particles may also be used in the formulation of structured dispersions. Strictly speaking, “particles” in dispersions are often viewed as the homogenous suspension of insoluble solids, and when they are dispersed in the surrounding “liquid phase” the resulting mixture is usually referred to as a “suspension”. When known suspensions become unstable, the particles “flocculate” and will eventually separate from the liquid phase and settle or sediment to the bottom of the containing vessel, forming a solid “cake”. “Droplets” in dispersions are commonly viewed as the homogenous suspension of immiscible liquids, and when they are dispersed in another liquid phase the resulting mixture is usually referred to as an “emulsion”. When emulsions become unstable, the smaller liquid droplets agglomerate and coalesce with one another on an accelerated basis, forming larger “globules” that will, sooner than later, separate from the dispersion—e.g., oil-water separation.

Depending on the application, the “PSD structuring” disclosed herein can involve the intentional manipulation of either the mean diameter or the large-diameter “tail” of the dispersion, or both, and this can be accomplished by deliberate (i.e., controlled) “shaping” of the particle (or droplet) size distribution, thereby producing new pharmaceutical entities. The “large-diameter tail” of a PSD refers to the population of particles larger than a certain “threshold” size, which in exemplary embodiments can be significantly larger than the center, or central tendency (e.g., mean, median or mode), of the PSD. By convention, the larger the extension, or deviation, of the “tail” toward larger particle sizes, the more “coarse” the dispersion is said to be. On the other hand, the term “shaping” of the PSD refers to the deliberate formulation of selected populations of particles or droplets of varying size(s) in the dispersion. In exemplary embodiments the resulting dispersions are relatively “stable”—e.g., they have relatively low rates of agglomeration of their constituent particles or droplets. “Stable” dispersions are characterized in, for example, Nicoli et al., 2006.

Therefore, the fact that a dispersion may be referred to as “coarse” is merely intended to be a relative description, consistent with the known characterization applied to traditional “fine” (i.e., non-“coarse”) dispersions, in which large-particle “tails” are substantially less extensive in both size and concentration (number/mL), and the dispersion more closely follows a log-Gaussian, or log-Normal, distribution with a well-defined mean diameter and standard deviation (SD). In the latter case, a “coarse” dispersion, implying the existence of a relatively extensive large-diameter tail in the PSD, is undesirable, because such a large-diameter tail usually implies instability with respect to increasing particle agglomeration, which is not the case for the structured dispersions described herein.

With exemplary dispersions disclosed herein, the shape of the PSD substantively deviates from normally distributed particle size values, instead resembling more a Pareto or power law distribution, having more emphasis on what would be considered “outlier” sizes in a normal distribution. In fact, exemplary embodiments involve the deliberate creation (i.e., addition) of one or more particle/droplet population peaks superimposed onto the large-diameter tail of the PSD. The peaks can be deliberately constructed in the present application and represent separate populations of particles or droplets/globules that remain stable—i.e., the sizes of the particles comprising the peaks do not grow appreciably in time and thus, the mean sizes of the peaks can be independent of the overall mean particle size (i.e. mean particle diameter) of the primary dispersion. This comparative example assumes that in a normal or log-normal distribution, the population of particles or droplets in a given tail is vanishingly small beyond a given large size, and therefore the PSD is tightly centered around a given mean size. By contrast, in a power law distribution there could be various clustered populations of droplets or particles superimposed on the tail, with possibly each clustered population forming a “sub-emulsion”, having a distinct, separate PSD compared to the PSD of the main, or “primary”, emulsion.

The term “active ingredients” as used herein refers to the therapeutic agents carried by particles or droplets that are intended to be delivered to one or more specific targets via a particle size-structured dispersion, such as a particle size-structured parenteral dispersion. The term “biodegradable” used herein refers to the metabolic fate of particles or droplets contained in such a parenteral dispersion following intravascular administration. A “biodegradable” particle or droplet is often one that is deformable and therefore able to squeeze through blood vessels smaller than its diameter. A “biodegradable” particle or droplet is also one that can be metabolized by the body, such as one composed of various carriers made from lipids or polymers with or without surface coatings. The rate of metabolism of the particle, droplet/globule or vesicle that carries the active ingredient(s), and/or the diffusion of said active ingredients from said particle, droplet/globule or vesicle can be modified. For example, modification of lipid droplet carriers may include altering the length of the hydrocarbon chain of the lipid used in the carrier or vesicle, or combining various mixtures of fatty acids or lipids of varying hydrocarbon lengths. In any event, the “residence time” in the patient's body of biodegradable particles or droplets/globules may, for example, be relatively short, such that their half-life will be correspondingly relatively short (compared to their shelf-life before injection), but it can be varied according to design.

Biodegradable particles or droplets/globules can act as carriers of active ingredients intended to be temporarily deposited in a targeted blood vessel or organ until they are broken down. In contrast, a non-biodegradable particle or droplet that functions as the active ingredient is one that can be both non-deformable and non-metabolizable and made of inert material, such as a synthetic polymer. Thus, the “residence time” of non-biodegradable particles or droplets/globules may be so long as to effectively render the latter permanent, or at least such that they possess a very long half-life (relative to biodegradables), but which, again, can be made to vary according to design. Non-biodegradable particles or droplets/globules can often act as “carriers” of active ingredient(s) but they may also function mechanically in order to permanently occlude blood vessels, and thereby induce localized ischemia and cell death. It is, however, possible that non-biodegradable particles, droplets, globules, or vesicles could serve a “dual-function” purpose that entails the delivery of active ingredients to a desired site in addition to occluding or ablating vessels. The foregoing are examples merely to illustrate potentially useful applications of particle size-structured dispersions as described herein without loss of generality intended or implied.

The general use of chemotherapeutic agents (e.g., antineoplastic drugs and antibiotics) for the treatment of disease is well established. For example, chemoembolization is a known medical therapy for the treatment of cancer. It is associated with the parenteral delivery of a “coarse”, extemporaneously prepared dispersion—i.e., one comprised of a significant concentration of relatively large particles or droplets/globules containing antineoplastic drugs and/or adjuvants, intended for intra-arterial administration. This dispersion presently contains particles or droplets/globules that span a wide size range and thus it would be identified as a “polydisperse” system. In the exemplary embodiments described herein, “chemoembolization” is intended to represent but one specific example of a wide range of therapies in which the parenteral dispersions described herein can be used for the delivery of any pharmaceutical drug(s) and/or adjuvant(s). A dispersion as disclosed in this example is said to be “coarse” in the sense that its PSD contains an unusually large number of particles or globules of unusually large size compared to those present in the known view of mean particle or droplet size in a Normal, or log-Normal, distribution, as found, for example, in traditional parenteral (injectable) emulsions. Independent of its relative coarseness, exemplary dispersions described herein should be sufficiently stable—i.e., with respect to significant agglomeration of the particles or droplets and consequent growth from its central tendency (e.g., mean or median diameter) over relatively short times—so as to be suitable for rapid, or “bolus”, intravascular administration. At the time of use, however, exemplary chemoembolization dispersions as disclosed can be prepared without additional surfactants, and thus over a longer period of time can be “unstable” with respect to particle, or droplet, agglomeration. Consequently, such dispersions can be structured to induce the occlusion of localized vessels as a result of the proclivity of smaller particles or droplets to agglomerate into larger droplets or globules at the intended site, thereby achieving “therapeutic” ischemia.

For example, by direct arterial administration of a chemoembolization dispersion in a manner as disclosed herein, a liver tumor may be exposed to very high concentrations of antineoplastic drugs, thus enabling the dispersion to deliver pharmacotherapeutic therapy more efficiently than by known intravenous delivery of the same drugs into the systemic circulation. As well, however, the surrounding vasculature (comprising normal and newly formed collateral vessels) supplying blood to the tumor site may also be selectively occluded via embolization of localized blood vessels by the over-size particles or droplets/globules contained in the dispersion.

It has been shown (Folkman, 1987) that once cancer cells establish themselves in the host (via a process known as tumorigenesis), continued growth of the tumor requires that it must initiate the formation of new blood vessels (via a process known as angiogenesis) by means of various “growth factors” (e.g., vascular endothelial growth factor). The pharmacological application of agents known as “angiogenesis inhibitors” is designed to inhibit endothelial proliferation and/or apoptosis, accomplishing this action at the molecular level. (Folkman, 2007). In contrast, exemplary embodiments disclosed herein exploit the deliberate structuring of the sizes of the droplets or globules comprising a dispersion so as to constitute new pharmaceutical entities. By specific structuring of the PSD, one may accomplish the same desired vascular inhibition by virtue of performing mechanically the effect achieved by pharmacological actions of angiogenesis inhibitors, as well as providing tumoricidal chemotherapy. In fact, the correct or precise structuring of the PSD in order to provide mechanical/physical action via embolization may, in many instances, turn out to be as important as the pharmacotherapy that, as currently applied, is specifically directed at the tumor. The application of the particle size-structured dispersions as described herein in a controlled and precise manner may yield a far more effective response and outcome for both therapeutic approaches in addressing the malignancy.

In certain cases of hepatocellular carcinoma, for example, chemoembolization therapy can be a significant treatment option. However, this therapy has also been applied in advanced cases of other neoplasms, such as pancreatic, oral and oropharyngeal cancers. A known technique of administration is referred to as trans-arterial chemoembolization, or TACE, where the chemoembolization infusion is a mixture of water-soluble components, such as aqueous antineoplastic agents (e.g., mitomycin and/or doxorubicin), extemporaneously combined with a non-aqueous radiographic contrast agent, such as ethiodized oil. The quality of the resulting “mixture” can be highly variable, depending on the mixing techniques, but in any case, it can be physically unstable with respect to particle or droplet/globule agglomeration over any extended period of time. Therefore, it is made immediately prior to use in patients and then quickly injected into the liver tumor via the hepatic artery, often under some form of radiographic guidance.

The goals of TACE are quite apparent in the treatment of cancer. However, it is important to note that the pharmaceutical effects of the dosage form resulting from the delivery of this therapy are highly variable, as such dispersions are not commercially available and therefore are, in effect, made individually prior to each use. The variable quality of the final dispersion is greatly affected by the skill of the person preparing the chemoembolzation regimens (i.e., physican, pharmacist or nurse), as well as the clinical experience associated with the application of this therapy. Little or no attention has been paid thusfar to measuring, let alone controlling, the PSDs of the parenteral infusions that have been made for use in chemoembolization therapy. Hence, there has been no attempt to correlate the particle or droplet/globule size distributions with the efficacy of the therapy. Although this therapy is generally recognized by oncologists to be a viable treatment option—e.g., when performed on newly diagnosed liver cancers in their early stages, or when used as a palliative therapy for advanced inoperable cases—it is well understood that the response is often highly variable (Kaido et al, 2007). It is likely that in addition to the pathophysiology of the cancer—i.e., tumor biology and staging—the “quality” of the final dosage form, as defined by the PSD, is also a major factor affecting the outcome of the treatment.

There are several extemporaneous compounding approaches that have been undertaken in order to achieve adequate mixing of the formulation, ranging from vigorous shaking and/or vortexing, to syringe-syringe mixing, to the use of ultrasonic probes for reducing the sizes of the particles or droplets/globules and/or deagglomerating them in an effort to improve very-short-term stabilization of the dispersion. The final preparation for infusion is then subjectively examined by visual observation for signs of “fluid separation”. These compounding maneuvers are not well controlled, use no additional surfactants to stabilize the dispersion, and do not include routine particle or droplet/globule size testing (i.e., particle size analysis) of the final formulation prior to infusion in a patient. Consequently, the quality of the final dispersion, as characterized by the PSD, is highly variable. The achievement of both longer-term stability and uniformity of the dispersion, optimized through structuring of the PSD of the final dosage form, would be highly desirable. Exemplary embodiments as disclosed herein can achieve these and other goals.

### SUMMARY

Exemplary embodiments disclosed herein are directed to a Drug/Adjuvant Delivery System (D/A DS) comprising: a liquid carrier; and a particle-size structured dispersion of solid and/or liquid particles suspended in the liquid carrier.

### BRIEF DESCRIPTION OF DRAWINGS

Other objects and advantages will become apparent to those skilled in the art upon reading the following detailed description of preferred embodiments, in conjunction with the accompanying drawings, wherein like reference numerals have been used to designate like elements, and wherein:

FIG. 1. Stylized plots showing magnified portions of the PSDs of a “normal” (reference) dispersion having a mean diameter of 0.5 μm and three structured dispersions formed from, or as an adjunct to, the latter by the addition of a large-diameter tail of successively higher embolic (EMB) thresholds of 5, 10 and 15 μm.

FIG. 2. Stylized plots showing magnified portions of the PSDs of a “normal” (reference) dispersion having a mean diameter of 1.0 μm and three structured dispersions formed from, or as an adjunct to, the latter by the addition of a large-diameter tail of successively higher embolic (EMB) thresholds of 10, 20 and 30 μm.

FIG. 3. Stylized plots showing magnified portions of the PSDs of a “normal” (reference) dispersion having a mean diameter of 5 μm and three structured dispersions formed from, or as an adjunct to, the latter by the addition of a large-diameter tail of successively higher embolic (EMB) thresholds of 30, 60 and 90 μm.

FIG. 4. Stylized plots showing magnified portions of the PSDs of a “normal” (reference) dispersion having a mean diameter of 10 μm and three structured dispersions formed from, or as an adjunct to, the latter by the addition of a large-diameter tail of successively higher embolic (EMB) thresholds of 60, 120 and 180 μm.

FIG. 5. Stylized plots showing magnified portions of the PSDs of a “normal” (reference) dispersion having a mean diameter of 5 nm and three structured dispersions formed from, or as an adjunct to, the latter by the addition of a large-diameter tail of successively higher characteristic structured size (CSS) thresholds of 50, 100 and 150 nm.

FIG. 6. Stylized plots showing magnified portions of the PSDs of a “normal” (reference) dispersion having a mean diameter of 10 nm and three structured dispersions formed from, or as an adjunct to, the latter by the addition of a large-diameter tail of successively higher characteristic structured size (CSS) thresholds of 100, 200 and 300 nm.

FIG. 7. Stylized plots showing magnified portions of the PSDs of a “normal” (reference) dispersion having a mean diameter of 50 nm and three structured dispersions formed from, or as an adjunct to, the latter by the addition of a large-diameter tail of successively higher characteristic structured size (CSS) thresholds of 300, 600 and 900 nm.

FIG. 8. Stylized plots showing magnified portions of the PSDs of a “normal” (reference) dispersion having a mean diameter of 100 nm and three structured dispersions formed from, or as an adjunct to, the latter by the addition of a large-diameter tail of successively higher characteristic structured size (CSS) thresholds of 600, 1200 and 1800 nm.

FIG. 9. Stylized plots showing magnified portions of the PSDs of a “normal” (reference) dispersion having a mean diameter of 0.5 μm and three structured dispersions formed from, or as an adjunct to, the latter by the addition at successively higher concentrations of a large-diameter tail, having an embolic (EMB) threshold of 5 μm

FIG. 10. Stylized plots showing magnified portions of the PSDs of a “normal” (reference) dispersion having a mean diameter of 0.5 μm and three structured dispersions formed from, or as an adjunct to, the latter by the addition at successively higher concentrations of a large-diameter tail, having an embolic (EMB) threshold of 10 μm.

FIG. 11. Stylized plots showing magnified portions of the PSDs of a “normal” (reference) dispersion having a mean diameter of 0.5 μm and three structured dispersions formed from, or as an adjunct to, the latter by the addition at successively higher concentrations of a large-diameter tail, having an embolic (EMB) threshold of 15 μm.

FIG. 12. Stylized plots showing magnified portions of the PSDs of a “normal” (reference) dispersion having a mean diameter of 0.5 μm and three structured dispersions formed from, or as an adjunct to, the latter by the addition at successively higher concentrations of a relatively narrow “peak” of relatively large particles, having an embolic (EMB) mean size of 5 μm.

FIG. 13. Stylized plots showing magnified portions of the PSDs of a “normal” (reference) dispersion having a mean diameter of 0.5 μm and three structured dispersions formed from, or as an adjunct to, the latter by the addition at successively higher concentrations of a relatively narrow “peak” of relatively large particles, having an embolic (EMB) mean size of 10 μm.

FIG. 14. Stylized plots showing magnified portions of the PSDs of a “normal” (reference) dispersion having a mean diameter of 0.5 μm and three structured dispersions formed from, or as an adjunct to, the latter by the addition at successively higher concentrations of a relatively narrow “peak” of relatively large particles, having an embolic (EMB) mean size of 15 μm.

FIG. 15. Stylized plots showing magnified portions of the PSDs of a “normal” (reference) dispersion having a mean diameter of 0.5 μm and three structured dispersions formed from, or as an adjunct to, the latter by the addition at successively higher concentrations of two relatively narrow “peaks” of relatively large particles, having embolic (EMB) mean sizes of 5 μm and 10 μm.

FIG. 16. Stylized plots showing magnified portions of the PSDs of a “normal” (reference) dispersion having a mean diameter of 0.5 μm and three structured dispersions formed from, or as an adjunct to, the latter by the addition at successively higher concentrations of a three relatively narrow “peaks” of relatively large particles, having embolic (EMB) mean sizes of 5, 10 and 15 μm.

### DETAILED DESCRIPTION

Exemplary embodiments disclosed herein exploit new physical criteria for the production and use of novel pharmaceutical dosage forms of stable parenteral dispersions based on deliberate, specific “size-structuring” of the particles or droplets/globules contained therein to form new pharmaceutical entities, and to optimize their effectiveness when used for the treatment of a variety of diseases. The resulting dosage forms thus produced are intended for, without limitation, intravascular administration via the intravenous or intra-arterial route. If the particle size-structured dispersion is administered intravenously, either via the peripheral (small vessel) or central (large vessel) venous circulation system, the primary target in this setting can be the lung, the proximal site where the infusate first passes. However, in the case of other distal vital organ target sites, such as the brain, heart, liver and kidneys, alternative methods of administration, such as intra-arterial injection of the size-structured dispersion to the organ, or arterial catheterization and retrograde direction of the catheter to the desired tissues, can be accomplished. The present disclosure relates to the formulation of particle size-structured dispersions as new pharmaceutical entities, and the use of parenteral dispersions that are “polydisperse” in particular, predetermined and desired ways—i.e., dispersions that contain particles or droplets/globules that span and emphasize various desired ranges of sizes. Such dispersions are deliberately designed to be “PSD-structured” in particular ways, so that the PSDs will possess desired “structures,” or shapes, for optimization of particular therapeutic purposes. There is particular, but not exclusive, emphasis on controlling or tailoring the shapes of the relatively coarse “tails” of large-diameter particles or droplets/globules of a given or desired PSD so as to optimize the targeted delivery of therapeutically active drugs and/or adjuvants used in the treatment of various diseases.

For example, the “structure” of a given large-diameter PSD “tail” could be customized to each given intended therapeutic use, and therefore the PSD adopted for each dosage form would be unique with respect to its specific application or indication. In addition, a given dispersion may also desirably contain a substantial population of relatively small (“fine”) particles or droplets for the purpose of addressing certain “systemic” effects, if indicated, which may be supplementary (i.e., adjuvant therapy) or complementary (i.e., synergistic) to the targeting of drug(s) to a particular organ or site and/or with causing occlusion of the surrounding blood vessels. Inclusion of this fine-particle “systemic” component in a dispersion is consistent with the existing use of conventional “fine” parenteral dispersions—i.e., for which the PSD has not been deliberately structured, or shaped, but rather conforms to a known Normal, or log-Normal (bell-shaped), distribution, where the mean particle or droplet size can be significantly smaller than one micrometer (1 μm).

Such an approach, for example, would be particularly relevant in the case of treating deep-seeded lung infections, where the organism has established itself within the organ and thereby may serve as a septic focus, but also where it causes a secondary (or metastatic) blood stream infection. In this case, it is desirable to attack the infection both locally and systemically. These two goals can be accomplished simultaneously by, for example, having one high dose of antimicrobial(s) carried by the size-structured large-diameter population of particles or droplets/globules comprising the tail portion of the PSD, designed to address a localized infection in the lung, but also having a second, lower dose of the antimicrobial agent(s) carried by the much smaller particle or droplet population, in order to address the remaining organisms circulating in the bloodstream. These latter organisms are possibly capable of producing metastatic infections in distant or satellite locations, independent of the primary infection emanating from the lungs. This is a particularly important embodiment of the disclosure, because currently the major limiting feature of many potent antibiotics is the toxicity associated with systemic exposure (e.g., nephrotoxicity, ototoxicity, etc.), related to very high serum drug concentrations. The current dilemma is based on the fact that the present therapy for major organ infections is usually limited to systemic administration of large doses of antimicrobials in order to produce sufficiently high serum drug concentrations flowing to the organ to be able to eradicate the infection in the target organ “indirectly”—i.e., via the circulatory system alone.

In this case the particle size-structured dispersions disclosed in the present disclosure would be applied to directing such therapy to the target organ safely in order to localize antimicrobial exposure, and thereby limit excessive systemic exposure and associated adverse drug-related events. It is also anticipated that more than one antibiotic may be incorporated within separate or possibly multiple peaks of the PSD of the size-structured dispersion, either when “mono-therapy” is not considered to be a reasonable therapeutic option or when the infection is caused by multi-drug-resistant organisms. Alternatively, through application of the novel drug delivery method described herein, it may also be possible to render that monotherapy more effective when administered in this unique manner. This approach may be especially important, for example, in the case of critically ill patients having multiple organ failure, where the risk of drug toxicity due to the use of other antimicrobial agents may outweigh the benefits of multiple drug therapies.

Similarly, the use of a combination of active ingredients in order to achieve local effects with limited systemic exposure for therapeutic indications may also be applied in other disease conditions, such as cancer. Such an approach would be directed at the primary tumor that may be localized in or near a major organ system, but for malignancies that have a high risk of metastasizing via the bloodstream and depositing in other sites, thereby producing secondary cancers. In this example, the PSD would be designed so that the population consisting of smaller particles or droplets carrying active ingredients would contain significantly lower amounts of active ingredients compared to the population of larger droplets in the particle size-structured dispersion containing the anticancer therapy. This “lower” dose of oncolytic(s), in sufficient concentrations, would be intended to address the circulating metastatic cells or collections of cells that have gathered together in satellite locations. The dose of oncolytic(s) delivered systemically in this manner could be sufficiently large to be tumoricidal, but still sufficiently small so as to drastically reduce serious adverse drugs events known to occur with the same drug(s) when given by known routes of administration at doses based on current formulations. The population of larger droplets would be locally directed to the primary tumor site. Whether these different populations of droplets carrying active ingredients are administered separately or within the same dispersion is based on both practical considerations (i.e., what is pharmaceutically and physiologically possible) and also therapeutic potential (i.e., depending on the desired clinical outcome).

In principle, just as infectious microorganisms or certain cancer cells often set up “residence” in or near a vital organ system, and then shed or metastasize to distant or satellite sites throughout the body, the PSD-structured dispersion techniques described herein can be similarly designed to follow the same “path” in order to combat the disease-producing cells, but in a highly controlled and organized manner. In particular, the “PSD-structuring” of the large-diameter tail of a dispersion can achieve desired localized effects, with or without emphasis on, or the inclusion of, a significant population of fine particles intended to address systemic effects. Alternatively, the PSD structuring may be designed to contain a very narrow range of sizes—i.e., a single “peak”—so that the distribution of particles or droplets/globules is substantially monodisperse and therefore focused on a particular size (or narrow range of sizes), compared to known, relatively polydisperse dispersions. Such particle size-structured dispersions are designed to contain stable and separate, but distinct, populations of particles or droplets/globules of varying size(s), where each population is associated with a discrete therapeutic purpose. In one example, a certain population of droplet sizes might contain the same active ingredient(s) as another population of different size(s), but with different concentrations of active ingredient(s) in the two populations, in order to address the disease condition both locally and systemically. In another example, one population of one particular size might contain entirely different active ingredients than another population of the same or different sizes, where the different ingredients are intended to provide independent and possibly synergistic therapeutic actions, in order to achieve an overall desired outcome. Ultimately, practice of the methods taught herein can lead to improved outcomes by, for example, improving the efficacy of various current or future parenteral therapies designed to deliver more precisely certain active ingredient(s) and thereby improve both their efficacy and safety profile.

In addition to chemoembolization or antimicrobial (also known as antibiotic) therapy, the particle size-structured dispersions will also be clinically useful as therapies for the treatment or ablation of vascular aneurysms. Here, the therapy is principally intended to deliver an embolic agent—i.e., one which achieves “blockage”, serving as a means of mitigating blood flow and reducing, or preventing altogether, the subsequent expansion of major vessels, which, in the absence of appropriate intervention, may result in the rupture of such vessels, leading to fatal hemorrhage. This is particularly true when surgical intervention cannot be safely applied. In this circumstance, intravascular administration of a selective embolic agent may be lifesaving. As the embolic agent is an important aspect of this therapeutic example, the desired “structure”, or shape, of the large-diameter “tail” portion of the PSD of the dispersion will be determined by the vessel(s) involved, and thus may require one or more “peaks” in the PSD in order to achieve the desired level of effective embolization. Thus, in the present context “peak” refers to the existence of a relatively high concentration of particles or droplets covering a relatively narrow range of sizes centered about the location (i.e., mean or median size) defining the peak. In this case, the particle size is selected to accomplish the desired occlusive effect, and the mean size of the population of particles or droplets/globules—i.e., the characteristic structured size (“CSS”, used hereinafter)—is dictated by the site or region of desired embolization.

For example, if capillary lodging or embolization is desirable, then the key structuring of the dispersion would focus on a peak comprising particles having a mean particle size of at least 5 micrometers (in diameter), such as about 5, 6, 7, 8, 9, or 10 micrometers. Approximately double this size, such as a mean particle size of at least 20 micrometers, would be used for larger vessels such as the arterioles and venules, and even larger sizes would be used for their respective terminal branches. About 1% or more on a mass or volume weighted basis of the total dispersed phase can be included in the peak. In exemplary embodiments, about 10% or more on a mass- or volume-weighted basis of the total dispersed phase can be included in the peak. In exemplary embodiments, the particles comprising the peak can have a size distribution wherein the size range of the distribution is about 20% or less of the mean particle size of the particles comprising the peak, such as about 20%, 10%, 5%, 2% and 1% of the mean particle size of the particles comprising the peak. In exemplary embodiments, the overall mean particle size of the particles in the dispersion is at least a factor of ½ log to 1 log below the mean particle size of the particles comprising the peak. As well, the composition of the active ingredient(s) and/or particles or droplets of the dispersed phase that function as an embolic agent may be important. If the size-structured dispersion is designed to induce therapeutic ischemia, then the embolic agent may desirably include a biodegradable set of ingredients. Suitable biodegradable materials include polymer esters such as, for example, the polylactides poly(L-lactide) or PLLA and poly(D-lactide) or PDLA. A primary function would be to occlude key blood vessels and thereby deprive life-sustaining oxygen and nutrients to a tumor, thus achieving apoptosis, or the staunching of blood flow, with the eventual biodegradation of the ingredients of the dispersion. Alternatively, the particles or droplets/globules used for embolization purposes may need to be constructed of non-metabolizable and non-deformable materials, such that they do not biodegrade and, instead, are designed for longer-term, or even permanent, occlusion, so as to effectively accomplish the same desired outcome. Suitable non-biodegradable materials include latex, polystyrene beads, and expanded polytetrafluoroethylene grafts (Gore-Tex). The need for a biodegradable or non-biodegradable dispersion would similarly be determined by the clinician and depend on the pathophysiology of the condition, its anatomical location and other clinical considerations.

Similarly, in another example, the parenteral dispersion may be structured with respect to certain desired particle or droplet sizes so as to allow delivery of the desired pharmacotherapy in the form of optimally-sized droplets that are capable of temporarily “lodging” at a desired target site and thereby “depositing” the dispersed active ingredient(s) there. Conceptually, it might be useful to view this aspect of the application in connection with chemoembolization and, by way of emphasizing its mechanical role, refer to it more descriptively as “chemodeposition”. The final dispersion is deliberately designed with respect to its distribution of particle or droplet sizes in such a way as to accomplish the desired pharmacotherapeutic effect, but at the same time specifically avoid significant occlusion or damage of vessels. Here again, the PSD of the structured particle or droplet population, as well as its composition, can be important in achieving the desired “depositing” effect. In this circumstance the capillary vessels may be of greatest importance, even though they account for only a small fraction of the total circulating blood volume, for it is within the capillary system where blood and tissues (i.e., target sites) meet. In this case the mean particle size of the particles or droplets in the peak should be in the approximate size range of 5 to 15 micrometers, such as about 5, 6, 7, 8, 9, 10, 11, 12, 13, 14 or 15 micrometers, so that they may “lodge” at the desired site.

The composition of the particles or droplets can be such that they are rapidly biodegradable, i.e., deformable and metabolizable, in order to avoid ischemic death by occlusion or embolization. The role of the size-structured dispersion in this case is to deliver the active ingredient(s) to the desired target, e.g., the lung, and allow the intravenously delivered particles or droplets to lodge there and gradually break down, permitting the antimicrobial(s) to diffuse into the infected tissues, thereby achieving the desired high, localized concentration of the drug. Thus, upon parenteral infusion, the desired pharmacotherapeutic action is accomplished primarily by delivering relatively precise concentrations (numbers) of specific, particle size-structured large-diameter, drug-containing particles or droplets/globules to the “target site”, so as to deposit the active ingredient(s) at that site in very high concentration, without necessarily producing embolization of the surrounding vasculature.

For example, in the treatment of pneumonias, the delivery of antibiotics to the infected site might be accomplished by intravenous injection of a dispersion structured to contain drug-laden globules (i.e., “over-size” droplets) having a predetermined range of sizes. Use of such a structured dispersion would be designed to deposit a given drug directly into the affected tissues, thereby producing a high local concentration effect, after which the globules would slowly biodegrade in a manner that avoids ischemia, while still providing high local concentrations of active ingredient(s). A globule population containing one or more peaks in the globule size distribution (GSD) may be designed so that various larger globules degrade at a different rate than other smaller droplets/globules that may be present in the same size-structured dispersion. In exemplary embodiments, multiple peaks in the GSD are designed so that the globules associated with each size peak biodegrade at different rates so as to maintain a high local concentration of drug over time.

Different size peaks in the GSD can be designed to perform different functions. Examples of specific applications, or functions, that may be achieved by the deliberate structuring of the GSD in this way include systemic delivery of drugs (i.e., throughout the entire circulatory system); local deposition of drugs; occlusion of blood vessels; achievement of multi-purpose action of systemic delivery of drug(s) combined with local deposition of drugs and/or occlusion of blood vessels; and other combinations of these aforementioned functions. In addition, the size-structured dispersion would presumably also contain a population of relatively fine (e.g. submicron) droplets that are too small to be able to “deposit” in relevant tissue or “occlude” blood vessels. These fine droplets would therefore have the function of providing a more conventional systemic effect, by releasing drug(s) throughout the body via the circulatory system, as with conventional pharmaceuticals. The ratio of the concentrations of particles or droplets/globules of each predetermined size to the corresponding concentrations of active ingredient(s) in a given formulated size-structured dispersion would be determined based on the therapeutic indication (as in the above example for lung infections). However, the desired ratio of the number (i.e., per unit volume of the dispersion) of particles or droplets/globules to the concentration of drug(s) for each particle or droplet/globule size in the size-structured distribution can also depend on the organ(s) involved, the type of infection (e.g., bacterial, fungal or viral) and the clinical prognosis.

Also included are dispersions that contain a single, relatively narrow peak in the PSD or GSD, where there is a much narrower range of particle or droplet/globule sizes than that encountered in a conventional injectable dispersion. The optimal choice of the peak particle or droplet/globule size can depend on the intended target and therapeutic goal of the parenteral dispersion.

Thus, it can be appreciated from the above description that multiple scenarios are possible for structuring the size distribution of the dispersion, where the best choice largely depends on its intended use. Numerous combinations of the particle or droplet/globule size(s) and possible desirable peak(s) in the dispersion may be devised. As well, the selected population(s) of particles or droplets/globules may be further manipulated so that, for example, they biodegrade with time at a single rate. Alternatively, in the case where there is more than one peak in the PSD or GSD of the structured dispersion, the particles or droplets/globules may be designed to biodegrade at different rates. Furthermore, the population(s) of particles or droplets/globules may be designed to function “mechanically” (i.e., by occluding blood vessels) or pharmacologically (i.e., by releasing drug(s)), or even in a hybrid manner (i.e., by combining both of the aforementioned functions) in order to produce the desired therapeutic effect(s). Hence, the use of numerous permutations and combinations of these physical and pharmacological effects may be possible through the efficient application of the methods taught in this disclosure, to be considered as giving rise to a new pharmaceutical entity.

The particle size-structured dispersions can be provided as a single formulation that combines one or more peaks in the PSD or GSD for the intended purpose(s) or, alternatively, given as separate infusions of dispersions having different PSDs or GSDs. In either case, a certain size-structured population of particles or droplets/globules is created as a stable dispersion. Then, the desired parenteral formulation may, for example, combine the separate dispersions into a single final dispersion, containing multiple populations of particles or droplets/globules. These populations would be distinctly different from one another, where each is associated with a particular function, or collection of functions, that may in some cases also act synergistically to address the underlying disease. Thus, the final formulation may have a superficial appearance of a coarse formulation, i.e., possessing a significant, extended large-diameter tail, but it will be designed to be a pharmaceutically stable dispersion by virtue of combining two or more stable components, or distributions. In this way, also, it will be possible to vary greatly the drug concentration contained in each particle or droplet/globule size that is present in the PSD or GSD of the dispersion. The particle-size structured dispersions described herein will apply to both new and existing drugs, and may improve drug delivery, efficacy and safety.

According to the Food and drug Administration (FDA), 2007 was a year in which one of the lowest number of applications for drug approvals were granted. On Dec. 6, 2007, the Wall Street Journal (US) article entitled Big Pharma Faces Grim Prognosis

stated the following when referring to the dearth of new molecular entities: “ . . . The industry estimates only one of every 5000 to 10,000 candidates makes it to human trials . . . But those odds seem to have worsened in recent years”, a reflection of the diminishing number of promising drugs in their respective pipelines. Consequently, many pharmaceutical companies are entering the generic drug business, most notably stated in the Sep. 24, 2008 Wall Street Journal (EUR) article entitled AstraZeneca Plans Shift Towards Branded Generics, where, for example, it stated: “ . . . Big pharmaceutical companies have been looking at ways to diversify their business into newer areas as they face the loss of patents covering their top-selling products as well as pricing pressure from health insurers and generic competitors in prime markets such as the U.S., Europe and Japan.”

As the drug industry transitions from a chemistry-based model (new molecular entities) of treating diseases towards a biotechnology-based (biologics license applications) drug development strategy, the compositions and methods described herein may offer an attractive and important alternative approach for drug development. Application of a physical-based approach (i.e., through the use of the present size-structured injectable dispersions), particularly involving complex disperse systems as new pharmaceutical entities designed to deliver active ingredients to specific target sites, may improve the clinical response to existing drugs for many diseases. Exemplary embodiments described herein can be used to optimize the delivery of active ingredients (i.e., drug(s)), whether for newly developed drug applications, or existing therapies. In this case, the design of particle-size structured parenteral dispersions will be an important, therapeutic adjunct to known drug-based therapies.

### 1. Exemplary Embodiments

The PSD-structured dispersions disclosed herein are new pharmaceutical entities. Under usual circumstances—i.e., for applications involving the use of injectable dispersions or emulsions other than those, such as chemoembolization, discussed herein—dispersions and emulsions are generally formulated so that they specifically possess a relatively high degree of “fineness” in the final commercially available product. Ideally, a “fine dispersion” is one in which the overwhelming majority of dispersed particles or droplets, expressed on a volume-weighted basis (or even more so on a number-weighted basis), are both relatively small (e.g., compared to the mean diameter of capillaries) and also lie within a relatively narrow range of sizes, as described quantitatively by the SD parameter of the PSD. In such cases the volume percentage of dispersed material contained in the coarse, large-diameter (“outlier”) particle or droplet tail of the PSD or GSD is vanishingly small, and usually seemingly insignificant. The recent pharmacopeial requirements on lipid injectable emulsions for globule size limits, e.g., <0.05% on a volume-weighted basis of particles or globules larger than 5 μm, illustrates the low tolerance for large-diameter fat globules in a formulation with an average mean droplet diameter of approximately 300 nm. (USP31/NF26, 2008—hereinafter referred to as USP <729>). Such dispersions are said to have a low index, or degree, of polydispersity, in describing the size range of the total population of particles or droplets in comparison to their central tendency, or in the case above, the mean droplet diameter. Given the large number of droplets per milliliter (mL) in such formulations (e.g., up to 10¹²/mL or more), the shape of the PSD typically resembles (approximately) a Normal, log-Normal, function. Injectable dispersions (e.g. lipid emulsions designed for parenteral nutrition) may sometimes fail this criterion—i.e., they may be excessively polydisperse, having a disproportionately high concentration of larger droplets or globules (and also often relatively unstable). In such cases it is the inordinately high concentration of agglomerated particles or droplets/globules, located at the upper size range, or the large-diameter tail, of the PSD or GSD that commonly produce formulations that fail pharmacopeial specifications (USP <729>).

Known pharmaceutical dispersions are specifically formulated to have a low degree of polydispersity, by limiting the breadth of sizes of the droplets or particles relative to their mean particle size (where the latter is also sufficiently small). By contrast, “coarse” dispersions, containing a wider distribution of particle or droplet sizes relative to their mean diameter, and usually also a relatively long, or “fat”, tail of abnormally large particles or globules, are commonly viewed as aberrant formulations—i.e., as batch failures. The adverse effects of uncontrolled (and usually undetected) coarsening of the PSD associated with poor-quality (and also often unstable) dispersions, as per conventional viewpoints, may be manifested in a variety of ways in the clinical setting. Drug toxicity or subtherapeutic response resulting from poor bioavailability, caused by inhomogenous delivery, can occur. Of equal clinical importance, potentially fatal fat embolism of major blood vessels and/or vital organs occurring from the use of unstable lipid emulsions is another example. The fineness, and the stability (against extensive agglomeration), of ionically-stabilized dispersions, emulsions and suspensions can be assessed by systematically reducing the height of the inter-particle potential energy barrier that keeps the charged particles or droplets separated and in suspension. Such deliberate, controlled reductions in barrier height cause an increase in the degree of coarseness of the dispersion, by virtue of accelerating the rate of growth of the large-diameter tail of the PSD, where the rate of growth depends on the underlying stability of the dispersion. Lower quality dispersions of products of similar, or even identical, composition will possess a reduced, and hence “defective”, barrier height with respect to a given “stress” challenge. When identified, the reduced barrier height (with resulting reduced dispersion stability) can be investigated further in order to identify the underlying factor(s) responsible, allowing them to be ultimately corrected. This increase in the rate of PSD tail growth can be monitored through the use of sensitive particle-sizing techniques, coupled with careful, systematic titration of the pH, counter-ion concentration or other related chemical variables affecting the barrier height, as described in U.S. Pat. No. 7,150,996 (Nicoli et al, Dec. 19, 2006), incorporated herein by reference in its entirety.

In contrast to the disclosure of Nicoli et al cited above, exemplary embodiments disclosed herein involve the use of novel parenteral dispersions that, by deliberate design, are structured in size in a particular way and to a particular extent (and possess sufficient stability) so as to constitute new and potentially very important pharmaceutical entities. More specifically, the novel “physical” design of dispersions taught herein is intended to exploit the distribution of particles or droplets/globules of such dispersions, through deliberate “size-structuring”, so that they can be used therapeutically in uniquely new and beneficial ways. The resulting distribution of particles or droplets/globules would necessarily deviate significantly in shape from the known (approximate) log-Normal, or Normal, function heretofore utilized. For example, one proposed approach for altering the PSD of a known dispersion involves restructuring the large-diameter tail, so that it exhibits a significant skewness toward the extreme sizes of droplet “outliers”, thereby producing a “fat tail” that is distinctly different from the tail of the PSD associated with known dispersions that approximate Normal, or log-Normal, distributions. Consequently, such specialized dispersions would traditionally be classified simply as being “coarse” and thus deemed to be undesirable, or “defective”, based on a traditional understanding of, and indication(s) for, ideal, “high-quality” dispersions. As already discussed, one important application of these novel dispersions is in the treatment of various cancers via an infusion method known as chemoembolization. This technique is presently used whenever the tumor is deemed suitably vulnerable, or it is deemed inoperable, and a reduction in tumor size followed by surgical resection is desirable, or alternatively, if the infusion is useful for palliative purposes. In this case, the therapy is designed to achieve two objectives: 1) targeted delivery of antineoplastic agent(s) directed against the tumor; and, 2) localized mechanical or physical embolization of those blood vessel(s) that support the viability of the tumor via transport of oxygen and nutrients.

Meta-analyses of the efficacy of chemoembolization from randomized controlled trials have concluded that, in general, the clinical studies are of sufficiently poor quality that drawing any meaningful conclusions regarding the effectiveness of this technique is precluded. Variabilities relating to clinical experience, patient selection, biology of the neoplasm, tumor volume or burden, cancer stage, etc. and other clinical issues contribute to the difficulty of making quantitative assessments of the efficacy of chemoembolization therapy as currently practiced. However, an even more important variability is likely the lack of controls in the extemporaneous preparation of injectable formulations used for chemoembolization. This “unknown” surely results in highly variable PSDs of the associated injectable dispersions, and therefore this uncontrolled factor may, in fact, be one of the most important variables in the current inability to determine clinical outcome. Notwithstanding this crucial current shortcoming, chemoembolization continues to be a popular and promising therapeutic option for patients, especially those afflicted with hepatocellular carcinoma. The attractiveness of improving the effectiveness of chemoembolization, given its serious shortcoming of being dependent on dispersions of limited, or even non-existent physical characterization, based on the deliberate, particle size- structuring of such dispersions, is a good example of one of the principal motivations for this disclosure.

Exemplary embodiments disclosed herein use novel dispersions containing active ingredients, e.g., drug(s) and/or adjuvant(s), wherein the PSD or GSD of the constituent particles or droplets/globules that carry, or contain, these active ingredients has been deliberately structured or designed and subsequently monitored and/or controlled. For example, the structure of the large-diameter tail of the PSD or GSD may desirably be chosen to possess a simple monotonic decay of the concentration (i.e., number per unit volume) of particles or droplets/globules versus their increasing size, such that the rate of decrease of the concentration with size is predetermined and monitored/controlled before use. Alternatively, the mean droplet size and the shape/extent of the large-diameter tail of the PSD or GSD may desirably be designed so as to include a single “peak”—i.e., a “unimodal” population of particles or droplets/globules that spans a relatively narrow range of sizes, embracing a particular, desired average size.

In yet another variation, the large-diameter tail of the dispersion may desirably contain more than one “peak”—i.e., a “multimodal” distribution, containing two or more populations of relatively high concentrations of particles or droplets/globules centered at two or more different sizes, where the multimodality of the PSD tail and the choice of specific peak sizes depend on the intended effects. The dispersion may also contain a significant population of very small droplets designed to traverse the systemic circulation, similar to the biopharmaceutical profile of traditional therapeutic dispersions (i.e., liposomal oncolytics), but which are present in much lower concentrations, or doses, in order to address lower tumor burdens (i.e., malignant cells), and which also contain other droplet or particle populations. For example, in one sense it may be desirable to have particles or droplets containing drug(s) and/or adjuvant(s) that become temporarily “embedded” within the organ containing the tumor, in order to achieve a high drug concentration in the immediate vicinity of the tumor. This function may perhaps be best achieved with an injectable dispersion that has been structured so as to contain a single “peak” in the PSD or GSD. The range of sizes of the particles or droplets/globules comprising this peak population in this case is important. If this dispersion is administered via intra-arterial injection, larger sizes in the PSD or GSD can be used than, for example, for particle size-structured dispersions designed to “lodge” or be deposited in relevant tissue if they are given by systemic venous circulation. In addition, the composition of the dispersed particles or droplets/globules should be such that they are able to deform and be metabolized by the body in order to avoid occlusion of blood vessels.

However, inclusion of another, higher-size “peak” in the same or separate PSD or GSD may also be desired in order to achieve the function of occlusion of blood vessels, with the goal of restricting the blood supply to the tumor. For this second “physical”, as opposed to “pharmacological”, function, it is not necessary that the particles or droplets/globules comprising the second, larger-size peak of the PSD or GSD carry drug(s) and/or adjuvant(s). Rather, it is simply desired that they possess this larger size, and be particles or droplets/globules with a unique composition, compared to the particles or droplets/globules comprising the first, smaller-size population of the PSD or GSD, designed to perform the first, “pharmacological” function, of the therapy. In this case, the particles or droplets/globules comprising the dispersed phase of this second population are not required to be deformable or metabolizable by the body. Such “multi-purpose” therapy may also involve the use of additional size-structured peaks in the PSD or GSD in order to improve the response of the disease to the dispersion. Ultimately, having the ability to produce size-structured dispersions that are both stable and size- and/or target-specific may improve the safety and efficacy of these parenteral therapies. This is particularly true for therapies that employ active ingredient(s) that possess a narrow therapeutic index (i.e., where the therapeutic dose is close to the toxic dose, such as with many oncolytic agents) via localized parenteral administration, thus reducing systemic exposure and associated toxicity.

It is also useful to consider the potentially useful therapeutic approach referred to herein as “chemodeposition”. The primary intent of this therapy is to temporarily “lodge” drugs or active ingredients at the target site, rather than occlude and embolize nearby blood vessels, as described above in connection with chemoembolization. Rather, this alternative therapy may be intended to be purely pharmacological in nature, i.e., to achieve high local concentrations of one or more drugs in the immediate vicinity of the target site, in order, for example, to eradicate deep-seeded infections (e.g., in major organs and bone). After release, the drugs biodegrade over time (i.e., they are deformable and/or biodegradable). Such a therapy may be particularly beneficial for immuno-compromised patients who fail to respond to conventional antimicrobial therapies. In this case, it may also be desirable to incorporate more than one structured “peak” in the PSD or GSD of the injectable dispersion, with location-selective lodging of particles or droplets/globules, based on their size, designed to occur at various sites along the vascular supply line and on into the organ of interest. For example, incorporation of smaller-size peaks in the PSD can be used to lodge particles or droplets in the smallest blood vessels, such as the capillaries (i.e., in the approximate size range of 5 to 10 micrometers (um)). Progressively larger-size peaks in the PSD or GSD can be employed to lodge these larger particles or globules in correspondingly larger vessels, such as at the level of the arterioles (i.e., in the approximate size range of 15 to 25 um).

As well, the dispersion could also possess the physical profile of a known “fine” emulsion by containing a population of significantly smaller particles or droplets intended to provide systemic effects—i.e., for release of drug throughout the circulatory system. However, in this case there will be significantly reduced amounts of drug needed, compared to known injectable drug suspensions that are formulated without regard to any specific “target site(s)”. For example, this therapy, if given intravenously, would seem most applicable in cases involving lung disease or infection, whereupon administration by veins of either the peripheral or central venous circulatory system may be used to take advantage of the first-pass “lodging” of particles or droplets/globules through the pulmonary circulation system. Once “lodged”, the particles or droplets/globules slowly biodegrade, allowing the gradual diffusion of the active ingredient(s) into the surrounding affected tissues and eventually into the systemic circulatory system. Depending on the need to also achieve effective drug concentration in the bloodstream in order to address systemic aspects of the disease, there may also be a need to include a separate population of small particles or droplets to accomplish this function. Alternatively, the diffusion of the active ingredient(s) from the lodged, biodegradable particles or droplets/globules into the systemic circulatory system may be sufficient to achieve the desired added systemic effects. For such therapies, the size-structured dispersion can be infused by intra-vascular administration, depending, for example, on the intended use, the target site, and the primary, secondary and/or collateral blood vessels supplying the organ. In some cases, radiographic confirmation of the occlusion of the desired vessel(s) may be obtained before, during and following the infusion. For this reason, radiographic contrast agents may also be included in the particles or droplets/globules comprising the size-structured dispersion. Again, depending on the intended use, it is also possible to administer the size-structured dispersion by the intravenous route, in addition to intra-arterial administration as previously described for other therapies.

In yet another application of this disclosure, it may be desirable to create a structured parenteral dispersion of varying particle size and/or composition, but in which all particles or droplets are intended to be submicron. Therefore, the purpose of this particle-size structured dispersion, by virtue of its sub-micron nature, has nothing to do, per se, with the “depositing” of active ingredient(s) and/or the occlusion of blood vessels. Rather, the dispersion is structured to contain various populations of particles or droplets that biodegrade at different rates. In this case, by modifying the particle size distribution and/or composition of the particles, droplets or vesicles that carry the active ingredient(s), the rate of biodegradation can be modified so as to cause breakdown at different rates, thereby achieving the pharmaceutical equivalent of a sustained release dosage form. The difference, compared to known therapies, is that such a structured sub-micron dispersion is designed to be administered intravascularly, as opposed to the known extravascular approach, such as achieved with controlled-release oral tablets or capsules via the gastrointestinal tract, transdermal delivery systems and intramuscular depot injections. An intravascular delivery system that can slowly biodegrade and thus achieve directly a sustained release of active ingredient(s), therefore extending its therapeutic half-life in the bloodstream, would be novel and highly desirable in the treatment of certain diseases. Such a dosage form could reduce the total amount of active ingredient(s) necessary to treat the disease, as well as reduce the risks associated with excessively high serum drug concentrations, and hence might improve the overall safety profile of the drug.

As reviewed above, the prevailing view of what constitutes an optimal dispersion is based on the premise that a desirable formulation is one that contains a relatively narrow PSD, having an approximately “log-Normal” population distribution of particles or droplets, with an appropriately small mean diameter (resulting, effectively, in a negligible “tail” of much larger, “over-size” particles). According to the known view, such a distribution would have a relatively small standard deviation (SD), or coefficient of variation (in addition to having a sufficiently small mean diameter) and thus would be said to be “optimized”, possessing a low degree, or index, of polydispersity. Parenteral infusions having substantially wider PSDs—i.e., those of significantly increased polydispersity—have been traditionally viewed as undesirable and would therefore generally be considered to be “failed” formulations. The importance of the large-diameter tail of the PSD with respect to the known view of dispersion stability is captured in the following statements in U.S. Pat. No. 7,150,996 (Nicoli et al, Dec. 19, 2006): “The most devastating effects of agglomeration or coalescence in a commercially prepared dispersion or emulsion often occur in a size range that is approximately ½ to 1 log larger than the mean particle or droplet size. This size range where significant changes occur is referred to as the large-diameter “tail” of the PSD and is usually of the greatest interest. This relatively remote population constituting the tail of the PSD for stable dispersions generally accounts for less (often substantially less) than 1% of the overall dispersed phase, on either a number- or volume-weighted basis.”

In contradistinction to this viewpoint, the present disclosure considers a completely different class of applications for injectable dispersions (including emulsions), for which an alternative, indeed opposite, point of view regarding the “quality” of the dispersion is now required. In the context of the present disclosure, the traditionally undesirable large-diameter tail of the PSD can, in fact, be deliberately structured and exploited in order to yield an optimal “coarse” dispersion, designed to contain as much as 10%, or even more, on a mass- or volume-weighted basis, of the total dispersed phase (particle or droplets/globules and associated active ingredient(s)) in order to achieve the desired effects. This unique methodology for prescribing what would traditionally be considered a “compromised”, or “failed”, dispersion—one that would clearly be considered undesirable for other, known applications, such as parenteral nutrition—is valuable whether the dispersion is intended to provide a pharmacological function (e.g., local release of drug) or a physical function (e.g., occlusion of blood vessels), or both. Of particular distinction, it should be noted that known dispersions that are determined to be highly polydisperse are viewed as undesirable formulations, at least in part because the population of particles or droplets comprising the large-diameter tail of the PSD would normally be viewed to be a consequence of instability of the primary dispersion (i.e., ongoing, accelerated coalescence of smaller particles or droplets into larger ones). In contradistinction to this perspective, any peaks occurring in the PSD or GSD of a particle size-structured dispersion as described in this disclosure have been deliberately formulated to be present (and stable), as opposed to being an inadvertent consequence of the instability of a failed formulation. Each population peak can exist separately from any other similarly formulated peak(s) present in the PSD, with little or no interaction between them and no resulting instability.

This disclosure therefore presents a contrary view of polydisperse dispersions designed for therapeutic use. In the context of the present disclosure they represent a desirable outcome by virtue of the fact that the resulting dispersions are structured in size characteristics as precisely as possible, in order to achieve unique effects and beneficial therapeutic outcomes. Thus, the present disclosure deliberately calls for the design, or “structuring” of dispersions (including, by definition, emulsions) in such a way that they become, in effect, “unconventional” and therefore constitute new pharmaceutical entities. This opposite, but desired, outcome is almost invariably counterintuitive to those skilled in the art of making parenteral dispersions (including emulsions and suspensions) for therapeutic administration. Consequently, the dispersions currently available for use in chemoembolization, for example, are relatively crude, being manually fabricated by extemporaneous and uncontrolled preparation techniques, and therefore have largely unknown, necessarily irreproducible, size characteristics. There is no commercial parenteral product that has been properly designed, size-structured, or size-characterized (i.e., by means of suitable particle size analysis) as an optimized, appropriately coarse dispersion for use in this important therapeutic application.

Particle or globule size analysis is, therefore, an important aspect in accordance with exemplary embodiments disclosed herein. The techniques and methods employed can produce accurate and reliable results for both qualitative and quantitative assessment of the PSD or GSD. Analysis technologies employing single-particle optical sensing (SPOS) techniques, desirably also including automatic sample dilution capabilities, can be used.

There are several techniques for particle size analysis that are able to provide, with varying degrees of accuracy and resolution, quantitative assessments of the PSD or GSD for the various structured dispersions described herein. Choice of the most appropriate technique can be dictated not only by the size range required for a given dispersion design, but also by the quality of results needed—specifically, the accuracy and, especially, the size resolution. Broadly speaking, the different techniques can be divided into two main categories—“ensemble” and “single-particle”.

In ensemble techniques, the detected signal to be analyzed consists of a superposition of the responses generated simultaneously by many particles, representing a large range of possible sizes (and concentrations). The raw data are “inverted”, or “deconvoluted”, using an appropriate mathematical algorithm to obtain the desired PSD, necessarily resulting in an approximation of the “true” size distribution. In single-particle techniques, the detected signal is produced by only one particle at any given instant of time. The desired PSD is obtained simply by calculating a particle size from each single-particle signal response and incrementing the accumulated particle count in the appropriate discrete (size) channel of a multi-channel representation of the PSD.

In exemplary embodiments, single-particle techniques can be preferred over ensemble methods because of the size resolution that they deliver, resulting in a PSD that most closely resembles the “true” size distribution. This preference can be evident for PSDs that deviate significantly from a simple, single-peak (“unimodal”) distribution, instead possessing significant “structure”, such as a “fat tail” of over-size particles extending well above the main population of particles. In the absence of that additional structure, the PSD could otherwise be adequately characterized by just two parameters—i.e., a mean diameter and standard deviation. The same limitation is even more evident in the case of a multimodal PSD, having one or more secondary peaks located apart (e.g., above) the center of the main population. PSDs that possess such relatively high degrees of structure, or “polydispersity”, typically demand, by definition, a sizing technique capable of providing a relatively high degree of resolution, in order for the details of the distribution to be accurately revealed. Of course, these are the kinds of PSDs that are associated with the “structured” dispersions that form the basis of this disclosure. Hence, all other factors being equal, single-particle sizing techniques will generally be favored over ensemble-type methods for reliable and accurate characterization of the “structured” dispersions proposed herein.

Therefore, an exemplary technique for characterizing the specialized dispersions described in the current disclosure is single-particle optical sizing (SPOS). SPOS sensors use a laser light source and associated optics to generate a thin, ribbon-like beam of light that passes through an optical flow cell, thereby defining an optical sensing zone, or “view volume”, through which fluid and suspended particles pass at an appropriate, controlled flow rate. The light intensity profile is approximately uniform over the cross section of the flow channel, resulting in a nearly uniform detector response to a particle of a given size passing through the view volume, regardless of its trajectory. A starting concentrated sample can be diluted before or during the measurement process, so that the particle concentration lies below the “coincidence limit” of the sensor. Particles spanning the detectable size range will then pass one at a time through the view volume. Attractive schemes for diluting the starting concentrated particle suspension are described in U.S. Pat. No. 4,794,806 (Nicoli et al, Jan. 3, 1989) and U.S. Pat. No. 6,211,956 (Nicoli, Apr. 3, 2001)

The SPOS sensors contained in the ACCUSIZER™ family of particle size analyzers (Agilent Technologies, Particle Sizing Systems Div., Santa Barbara, Calif.), for example, utilize a proprietary combination of two known physical detection methods, as described in U.S. Pat. No. 5,835,211 (Wells et al, Nov. 10, 1998). The first detection method is that of light extinction (LE), in which passage of a particle through the view volume results in a momentary reduction in the light flux exiting the flow cell and impinging on a distant LE detector. The height of this negative-going pulse in the LE signal increases monotonically with the particle size. For the ACCUSIZER™ Model LE-400SE sensor, the LE-only response of the sensor has a practical lower detection limit of approximately 1.5 micrometers (um), resulting in a nominal size range of 1.5 to 400 um.

The second detection method is that of light scattering (LS), in which passage of a particle through the same view volume results in a momentary burst of scattered light that is captured over a particular solid angle by a second, LS detector. The height of the resulting positive-going pulse in the LS signal also ideally increases monotonically with the particle size. Use of the LS detection method results in a significantly smaller detection limit than that provided by the LE method—typically approximately 0.5 um. The combined LE+LS response described in the Wells et al U.S. Patent cited above results in an SPOS sensor having the merits of both an extended lower detection limit and relatively large upper size limit -a nominal range of 0.5 to 400 um for the sensor identified above.

Improvements in the sensitivity and coincidence limit of SPOS sensors have recently been made using a radically different optical and signal processing approach, based on the use of a focused light beam, as described in U.S. Pat. No. 6,794,671 (Nicoli et al, Sep. 21, 2004) and U.S. Pat. No. 7,127,356 (Nicoli et al, Oct. 24, 2006). Because the incident beam illuminates only a small portion of the cross section of the flow cell, only a correspondingly small fraction of the particles flowing through the sensor are detected. Because the incident light intensity field is no longer approximately uniform, but instead is highly focused, having an approximately Gaussian profile, the resulting LE or LS signal pulse height depends not only on the size of a given particle, but also on its trajectory with respect to the highly non-uniform, focused view volume. The resulting pulse height distribution (PHD) for particles of a given, uniform size is no longer narrow, as for an LE or LS sensor of known design, but now broad, and hence it must be deconvoluted to account for all possible particle trajectories in order to obtain the desired PSD. It should be noted that the use of a “deconvolution” process to remove the influence of the particle trajectory on the PHD does not per se reduce the resolution of the resulting PSD. The new focused-beam sensors discussed above still represent a form of SPOS—i.e., the signal at any given instant of time is still produced by only one particle. This is in sharp distinction to the ensemble techniques discussed below, where particles of all sizes contribute simultaneously to the measured “signal. There, an entirely different “deconvolution” process is used, and the resulting PSD suffers from significantly reduced resolution compared to that obtained from focused-beam SPOS.

Notwithstanding these technical issues, the resulting focused-beam SPOS sensors—the ACCUSIZER™ “FX” (LE version) and the ACCUSIZER™ “FX-Nano” (LS version)—possess two distinct advantages over their conventional known LE- and LS-type counterparts. First and foremost, they have much higher sensitivity—i.e., substantially lower detectable size limits than the values that are achieved from known LE- and LS-type sensors: approximately 0.6 um for the “FX” (LE) sensor and about 0.15 um for the “FX-Nano” (LS) version. The lower size limit provided by the FX-Nano sensor, in particular, may be invaluable for characterizing the PSDs associated with some of the structured dispersions discussed herein. The second advantage of the new focused-beam sensors is that the working particle concentration can be much higher than that of a conventional known LE or LS sensor—e.g., 1,000,000/mL for the “FX” sensor and 10,000,000/mL for the “FX-Nano” version, compared to only about 10,000/mL for a sensor, whether LE- or LS-type, utilizing a traditional light beam profile. Hence, starting concentrated samples involve much less dilution, thus simplifying the fluidics system required for the dilution process. However, of greater practical significance is the related fact that the new sensors can be much less sensitive to the background level of particulate contamination in the fluid used to suspend and dilute the starting sample. This feature is particularly valuable when the LS version of the FX-Nano sensor is used to probe the lowest limits of particle size, below 0.2 um. A conventional known LS-type sensor would require enormous dilution of the starting sample and prodigious, time-consuming filtering of the diluting liquid in order to achieve acceptable signal/noise ratios and thus “clean”, reproducible PSD results.

There is another single-particle sizing technique that, in theory, represents a viable alternative to the SPOS approach—the “resistive pore” method, historically referred to as the “Coulter Counter”. Analogous to the LE version of the SPOS technique, it is based on electrical conductivity rather than light intensity. This known method detects the momentary decrease in conductivity (i.e., increase in electrical resistance) between two bodies of partially conducting fluid connected, fluidically and electrically, by a small aperture, when a single particle passes through the latter due to a pressure differential applied between the two fluid bodies. The height of each negative-going pulse in conductivity provides a measure of the particle volume, and hence size, and, as in SPOS, the pulse height increases monotonically with increasing particle size.

Originally developed for determining the size distribution of red and white blood cells, this technique can be less well suited than SPOS for determining the PSD of relatively polydisperse samples, such as the structured dispersions proposed in the current disclosure. First, it possesses a relatively small dynamic size range for a given aperture size, because of inherent signal/noise limitations, due to “Johnson noise”, for particles smaller than approximately 1/10 of the pore diameter. Hence, in theory, several different aperture sizes would be used to cover the wide size range offered by the SPOS technique (especially when it utilizes the combined LE+LS method outlined in the U.S. Patent of Wells et al cited above). However, in practice the resistive pore technique is a poor choice for analyzing an especially broad PSD. Either the lowest-size region of the PSD will be missing if the aperture chosen is sufficiently large to be able to accommodate the upper end of the PSD, or the aperture will clog frequently if it is sufficiently small to be able to detect particles in the lower region of the PSD.

A second, more significant disadvantage of the resistive-pore technique is that the resulting PSD has much poorer counting statistics than a corresponding PSD obtained by SPOS for the same data collection time. Although the starting particle concentration, after appropriate dilution, can be significantly higher for a resistive pore apparatus, the flow rate through the aperture is much smaller than that employed for SPOS sensors—e.g., less than 1 mL/min, compared to 50-100 mL/sec for SPOS. Therefore, the data collection rate for SPOS can be at least 40-50 times higher than the rate that can be achieved using the resistive pore method (in both cases avoiding coincidence-related distortions in the PSD). Hence, the number of particle counts collected in a given size channel over a given time interval using the resistive pore method can be much smaller than the corresponding number generated by SPOS. Consequently, the statistical fluctuations, assuming the Poisson square-root law, will be much larger on a fractional basis for PSDs generated by the resistive pore technique than by SPOS. This difference is especially significant in the large-particle region of the PSD of structured dispersions, including over-size “long tails” and/or secondary peaks. In either case the percentage of particles on a number-weighted basis is usually relatively small to begin with in the large-particle region and, in any case, that percentage in general decreases with increasing size, thus enhancing the influence of statistical fluctuations.

In conclusion, in order to maximize the resolution and reproducibility of PSD results, which are invariably influenced by the relative level of statistical fluctuations, it is evident that, in exemplary embodiments, the SPOS technique can be more suitable than its resistive pore counterpart for characterizing the structured dispersions contemplated in this disclosure. Selection of a conventional SPOS sensor (based on LE, LS or LE+LS) or a focused-beam variant (“FX” or “FX-Nano”) depends on a number of considerations. These include the complexity of the PSD to be analyzed, the dynamic size range needed, the extent to which the starting sample needs to be diluted and the resulting complexity of the fluidics system required to perform the dilution. In any case, the SPOS technique is suited to the characterization of the structured dispersions defined in this disclosure, both in the early stages of product formulation and also later for quality control monitoring during large-scale manufacturing.

Alternatively, ensemble techniques have also been applied to PSD determination with varying degrees of success, depending on the complexity of the distribution. However, as noted above, they lack the size resolution necessary for ascertaining the detailed shape of the PSDs associated with many of the structured dispersions proposed herein. The most widely used ensemble technique is usually referred to as laser diffraction (LD). In reality it is based on two distinct physical principles—Mie scattering (MS) and Fraunhofer diffraction (FD). Use of the MS method is appropriate only for relatively small particle sizes—approximately 1/10 to 10 times the laser wavelength, or roughly 50 nm to several microns. It is based on the fact that the intensity of monochromatic (laser) light scattered by a particle in that size range varies with the scattering angle due to intraparticle interference, as described by Mie theory. For particles in the “Rayleigh” region, much smaller than the laser wavelength, the scattered light pattern is nearly isotropic—i.e., there is no significant angular dependence of the scattered intensity.

In principle, the angular “signature” of the scattered light intensity can be used to determine the size of particles in the Mie size region. For particles at the lower end of this range—i.e., substantially smaller than the wavelength—the intensity increases only slightly with decreasing angle, from the backward direction (180-deg) to the near-forward direction. As the particle diameter increases, the extent of angular “dissymmetry” increases progressively, eventually becoming non-monotonic with changing scattering angle. The pattern of angular dissymmetry is a function not only of the particle size, for a given laser wavelength, but also of the refractive index of the particle (as well as that of the suspending liquid), requiring knowledge of both the real and imaginary (absorption-related) components of the latter.

In practice, however, the MS method is much less useful when confronted with a PSD that is relatively broad, especially one that has significant “structure”. The pattern of variation of the scattering intensity with the scattering angle will vary with the particle diameter. These different patterns, each for particles of a different size, are added together with a weighting based on the concentration of particles of a given size, in order to obtain the observed variation of scattering intensity with angle. Of course, determination of the PSD involves the opposite process—i.e., the measured variation of scattering intensity with angle must be “inverted”, or “deconvoluted”, in order to obtain an approximation of the relative concentration of particles of each relevant size. This process is known to be “ill conditioned”, inherently having quite limited size resolution, and therefore poorly suited for characterizing important features of polydisperse PSDs, such as long, asymmetric “tails” of over-size particles and multiple (especially closely-spaced) peaks—i.e., the kinds of “structure” inherent in the specialized dispersions proposed in this disclosure.

A similar limitation, inherent in the deconvolution process, unfortunately also exists when the FD method is applied to polydisperse PSDs for particles above the Mie region, significantly larger than the laser wavelength—i.e., larger than several micrometers. In the idealized case of uniform particles, the signal response for the FD method is very simple, consisting of a set of diffraction “rings” of alternating intensity maxima and minima distributed over a range of small angles in the forward direction. The larger the uniform particles, the smaller the angular spacing between adjacent diffraction rings. In cases of interest, however, there is a range of particle sizes present. The forward-angle diffraction pattern then loses its periodic structure when intensity patterns of different periodicity, contributed by particles of different size comprising the PSD, are superimposed. Determination of the underlying PSD requires deconvolution of the measured diffraction pattern, as in the MS method. Similarly, the resulting computed PSD possesses limited resolution and as well frequently contains serious artifacts and distortions.

In conclusion, principally because of limited resolution and associated accuracy, the ensemble methods of Mie scattering and Fraunhofer diffraction, whether used individually or in combination (depending on the particle size range of interest), would be poorly suited for characterizing many, if not most, of the “structured” dispersions that form the basis of the current disclosure. This conclusion is supported by a relatively recent study (Driscoll et al, 2001) of the effectiveness of light obscuration (i.e., SPOS-LE) vs laser diffraction (i.e., MS) instruments for particle size analysis of the large-particle “tails” of unstable parenteral lipid emulsions.

It is worth noting that there is another ensemble-type approach that uses Mie scattering for particle size analysis. Rather than measuring the variation in light scattering intensity as a function of scattering angle at a fixed wavelength, this alternative method measures the change in optical absorbance of an appropriately dilute particle suspension as a function of the illuminating wavelength. The optical absorbance vs wavelength data, obtained using a scanning spectrophotometer, is again deconvoluted using an appropriate algorithm (and accurate knowledge of both the real and imaginary components of the refractive index of the particles and suspending liquid) in order to obtain an approximation of the desired PSD. It is believed in some quarters that this alternative approach to applying MS theory to PSD determination is able to provide results of superior accuracy and resolution compared to the traditional multi-angle MS approach discussed earlier. If this assertion is true, this alternative method may prove to be useful for characterizing the PSDs of some of the “structured” dispersions discussed herein. However, in any case it should be clear that single-particle methods—particularly the SPOS technique—may prove to be superior than MS-based methods for use in connection with this disclosure.

Finally, there is another known ensemble technique for determining the PSDs of particles in liquid suspension, but one that is effective primarily/only at the smallest end of the size scale—from a few 10s of nanometers to only 2-5 micrometers. This is the technique of dynamic light scattering (DLS), also known as photon correlation spectroscopy (PCS) or quasi-elastic light scattering (QELS). This technique, using a laser light source, analyzes the temporal fluctuations of the scattered light intensity caused by the changes in the relative phases of the scattered waves originating from each particle as they undergo random-walk Brownian motion, or diffusion, in liquid suspension. As described by the Stokes-Einstein formula, the rate of diffusion is inversely proportional to the diameter of the (spherical) particles, independent of their composition.

Quantitative information regarding the particle size distribution (PSD) can be obtained from the apparently random fluctuations, or “noise”, in the scattered light intensity detected at a particular scattering angle by electronic construction of its autocorrelation function (ACF). However, in general the DLS technique is inherently capable of yielding PSD results of only limited resolution. Like the MS or FD methods, DLS is an ensemble technique, requiring mathematical deconvolution of its signal “signature”—in this case the time-decaying ACF—in order to obtain the desired PSD. In practice, the success of such computations in providing an acceptable level of accuracy and, particularly resolution, of the resulting computed PSD varies greatly with the complexity and size range of the underlying distribution. In the case of a relatively narrow distribution, obtaining an accurate measurement of the mean diameter and width (i.e., the standard deviation (SD), or coefficient of variation (CV)) of the PSD is straightforward. However, in exemplary embodiments the “structured” dispersions described herein involve relatively broad, non-symmetric and “complex” PSDs—i.e., those that deviate substantially from simple, unimodal distributions. In these cases, the mathematical inversion techniques associated with the DLS technique may not be able to provide PSD results having the necessary resolution—e.g., able to delineate accurately an extended large-diameter “tail” or a deliberately-added secondary peak, to say nothing of multiple peaks, situated in the large-size end of the relevant particle size range.

In closing this summary of the DLS technique, it is worth noting that it is effective in characterizing PSDs that are primarily in the submicron size region. However, most of the “structured” dispersions described herein contain a significant population of particles, or droplets, larger than 1 um, and often larger than 5 um. This is the range of particle size that is least well measured using the DLS technique. Given the fact that the SPOS technique is able to be very effective in characterizing PSDs that extend down to 0.5 um (and substantially lower using the new FX-Nano technique), it is a moot point as to whether the DLS technique might be useful for characterizing the “structured” dispersions discussed herein.

In conclusion, SPOS is suited for determining the desired details of most of the structured dispersions discussed in this disclosure. However, in cases where there is a significant population of very small (“deep-submicron”) particles, inaccessible by any SPOS method (including “FX-Nano”)—e.g., smaller than about 100 nm—it may be useful to combine an ensemble technique, such as DLS, with SPOS. Such a combination will provide the greatest amount of useful quantitative information regarding the underlying, highly polydisperse PSD. Clearly, those skilled in the art will recognize the utility of various sizing techniques (whether used alone or in combination) as tools for creating the desired particle size-structured parenteral dispersions for optimized disease treatment.

In exemplary embodiments, the application of the aforementioned particle sizing methods can be central to the success of the optimization of the formulation, production and utilization of structured parenteral dispersions as described in this disclosure. Although the detailed choice of formulation of a dispersion system (i.e., choice of the active ingredient(s), aqueous and non-aqueous phases, preparation steps, etc.) is very important, confirmation that the desired PSD has been obtained is potentially equally, or even more, important in order to achieve the therapeutic goals described herein. The analysis techniques reviewed above are applicable to a wide range of applications, including characterization of the particle size-structured dispersions described herein. This review is intended to illustrate the need for the acquisition of critical, reliable information relating to the PSD in order to achieve the desired safe formulation. Other analytical techniques and methods not specifically mentioned in the review above may achieve similarly important information, and hence they may be possible candidates and applied accordingly. Thus, the selection of the best technique (and corresponding instrument) for particle size analysis will be determined by those skilled in the art, assuming that it yields vital information regarding the structuring of the parenteral dispersion as described herein. Therefore, no loss of generality is intended or implied with respect to the selection of an appropriate technique and instrumentation for particle size analysis and this choice is understood to be within the spirit and scope of the disclosure.

Such new therapies based on the use of particle size-structured dispersions as described herein are generally viewed as constituting “innovative” clinical care, particularly appropriate for use in desperate clinical situations. Therefore, as such, they would be relegated to secondary use relative to known methods of treatment, such as surgical resection and/or traditional “systemic” parenteral drug regimens. For example, the success of chemoembolization is at present unresolved, based on current literature reviews, and it is largely considered to be effective only when used as a palliative remedy, to be resorted to only when known treatment options are either not possible, or have previously failed. Exemplary embodiments disclosed herein can standardize, as well as make more effective, the formulations used in chemoembolization therapy, by deliberately specifying and controlling the “structure” of the size distribution of particles or droplets comprising the dispersion—i.e., the mean diameter and the size range and shape of the relatively large-diameter tail of the PSD or GSD. Implementation of this method of “physical” (as opposed to “pharmaceutical”) dispersion design can improve outcomes and possibly shift this therapy from its current position as a secondary therapeutic intervention towards a primary mode of treatment, offering superior clinical outcomes. Thus, such structuring of the PSD may not only improve the quality and consistency of the final formulation, and very likely the resulting clinical response, but it may also make the therapy safer, especially for drugs that possess a narrow therapeutic index.

Described herein are methods for prophylactic or therapeutic, transient or permanent, embolization, chemoembolization, and chemodeposition in a patient which comprise administering to a patient in need of such treatment a composition, particularly an injectable composition, comprising an effective amount of a particle-size structured dispersion. In exemplary embodiments, the patient is a mammal, such as a human. Examples of conditions and disease states that can be prevented or treated by the present methods are described herein and include, but are not limited to, solid tumors, vascular malformations, and hemorrhagic events or processes. Regarding tumors, the present methods can be used to suppress pain, to limit blood loss occurring during surgical intervention, or to bring on tumoral necrosis and to either avoid or minimize the necessity of surgical intervention. With respect to vascular malformations, the present methods can be used to normalize the blood flow to “normal” tissues, to aid in surgery and to limit the risk of hemorrhage. For hemorrhagic events or processes, the present methods can be used to reduce blood flow and to promote cicatrization of the arterial opening(s). In addition, the present methods can be used as a pre-surgical treatment in order to decrease the blood flow in blood rich organs (e.g., the liver) prior to surgical intervention. Examples of specific conditions that can be prevented or treated by the present methods include, but are not limited to: uterine tumors or fibroids; small intestinal hemorrhage, such as that associated with stress ulcer; surgical drain; anastomosis; tuberculous ulcer and nonspecific ulcer; symptomatic hepatic arteriovenous malformation (AVM); primary colorectal cancer; hepatocellular carcinomas; liver metastases; bone metastases; melanomas; cancers of the head or neck; and intracranial meningiomas.

The magnitude of a prophylactic or therapeutic dose of the dispersions described herein can vary with the nature of the type, location and severity of the condition to be treated and the route of administration. It can also vary according to the age, weight and response of the individual patient. Effective amounts of the dispersions to be used in the present methods can be based on the recommended doses known to those skilled in the art for the various conditions, diseases or disorders. An effective amount refers to that amount of particle-size structured dispersion sufficient to result in amelioration of symptoms or a prolongation of survival in a patient, sufficient to permanently or temporarily occlude the vascular lumen in question; and/or sufficient to result in local and/or targeted release of an active agent, such as a drug. Toxicity and therapeutic efficacy of such particle-size structured dispersions can be determined by standard procedures in experimental animals.

Any suitable route of administration may be employed for providing the patient with an effective dosage of particle-size dispersions as described herein at the desired target or location, including intra-arterial and intravenous administration. In exemplary embodiments, administration can comprise, without limitation, delivery inside targeted arteries and/or veins via a catheter.

The term “large-diameter tail” is, necessarily, relative, having specific quantitative meaning only in relation to the value of the mean diameter of the PSD. The intended uses of the chemoembolization and chemodeposition regimens will determine the most desirable and unique “structures” of the respective PSDs based on anatomical and therapeutic considerations, as described above.

The result of structuring the PSD, in general, and the large-diameter tail, in particular, of dispersions intended for clinical use therefore yields an entirely unique type, or class, of dispersions, in which the distribution of particles or droplets/globules may desirably span a wide size range, from nanometers to micrometers, and where the dispersions exhibit longer-than-usual, but in any case sufficient, shelf-life stability. There is an approximate “log-distance”—i.e., factor of 10—between the mean particle or droplet size and the beginning of the size range defining the large-diameter tail. For the sake of conceptual simplicity, it is useful to consider the particle or droplet/globule population as being represented approximately by the known Normal, or log-Normal, distribution, as discussed above in Nicoli et al., 2006.

Table 1 provides several examples of dispersions having various mean particle sizes (“MPS”), with the corresponding size ranges in the structured large-diameter tail, covering both a larger “micrometer” size range (0.5-10 um) and a much smaller “nanometer” size range (5-100 nm). The examples shown in Table 1 are useful for illustrating some possible particle size-structured formulations of varying polydispersity. Examples of lower and upper size limits for each large-diameter tail (“LDT”) are shown in Table 1, labeled as “LDTmin” and “LDTmax”, respectively. Of particular interest is the relationship between the size range for each LDT, ranging from LDTₘᵢₙ and LDTₘₐₓ, and the selected mean particle diameter for the entire distribution, represented by MPS. The size range where “structuring” of the large-diameter tail may be appropriate, or proposed, depending on the intended use for each dispersion, is also presented in Table 1 for each example.

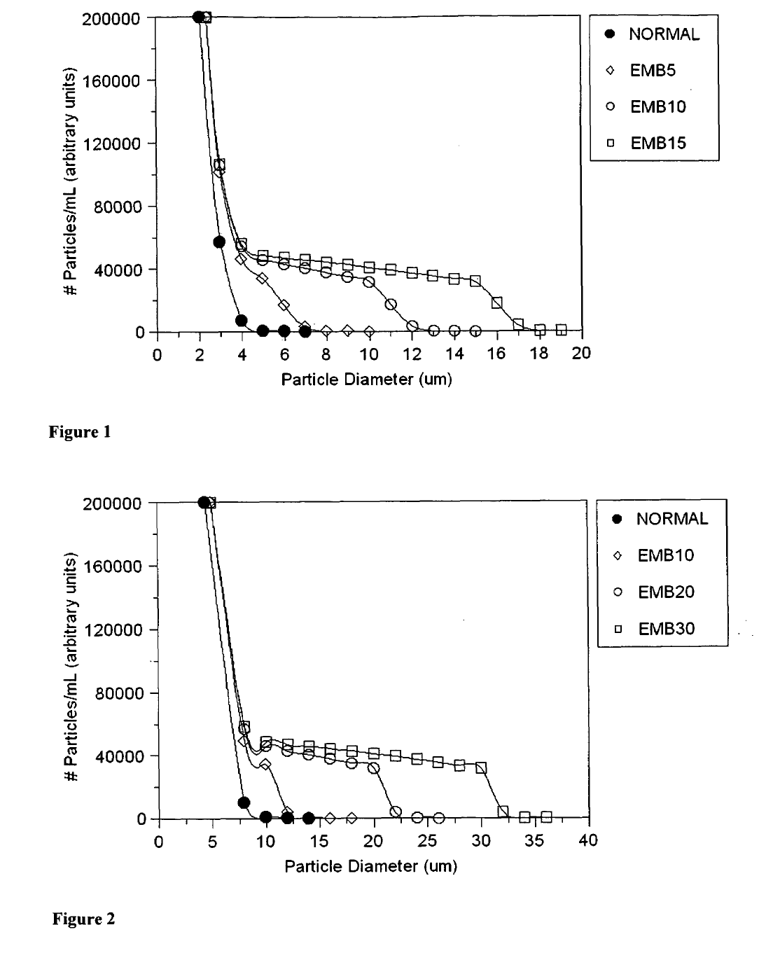

FIGS. 1-8 show stylized (i.e., idealized) plots of the LDT portion of the PSDs corresponding to the examples shown in Table 1, depicting some possible “structures” that may desirably be produced for the population of larger size particles or droplets/globules in the tail of the distribution. In each figure, for reference purposes, a large-diameter tail for a “Normal” dispersion—i.e., a known, “fine” dispersion of low polydispersity—is also plotted, indicated by closed black circles. This tail, decaying relatively rapidly in number/mL vs particle diameter, is then easily compared to the size-structured tails that extend out further in particle size, having increasing characteristic sizes, or “embolic thresholds” (EMB). Although the term “EMB” is used here, it could as easily be replaced by the term “deposition threshold” (“DEP”), depending on the intended use. There is no loss in generality intended or implied by the use of the examples shown in FIGS. 1-8. The terms, units and size ranges used here are merely illustrations of the size-structuring possibilities inherent in this application.

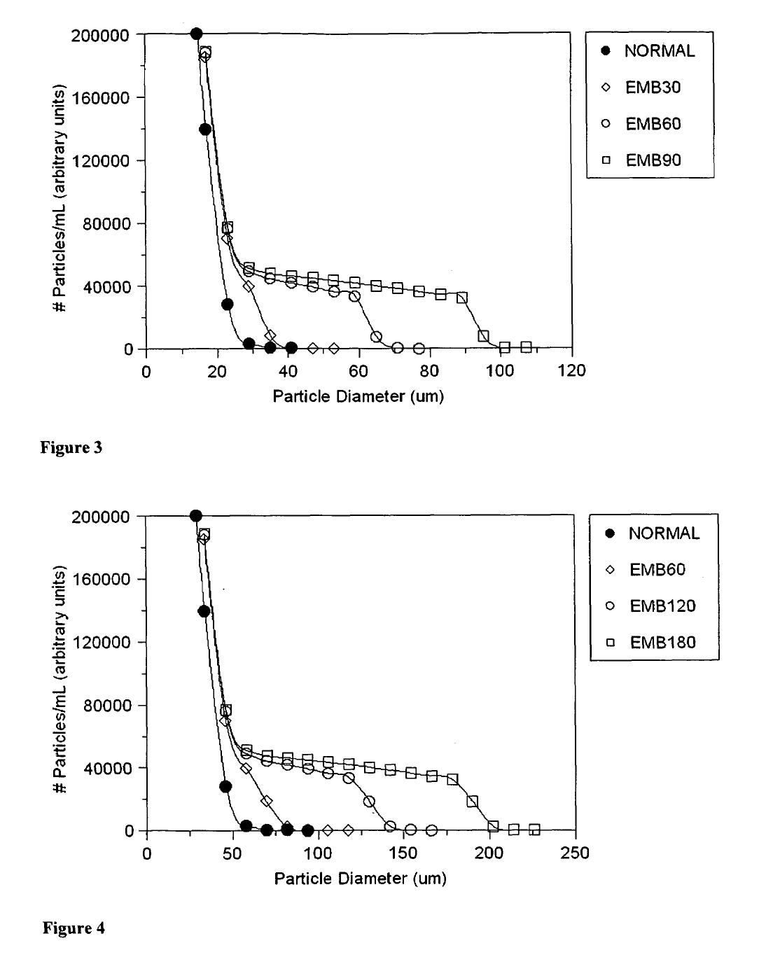

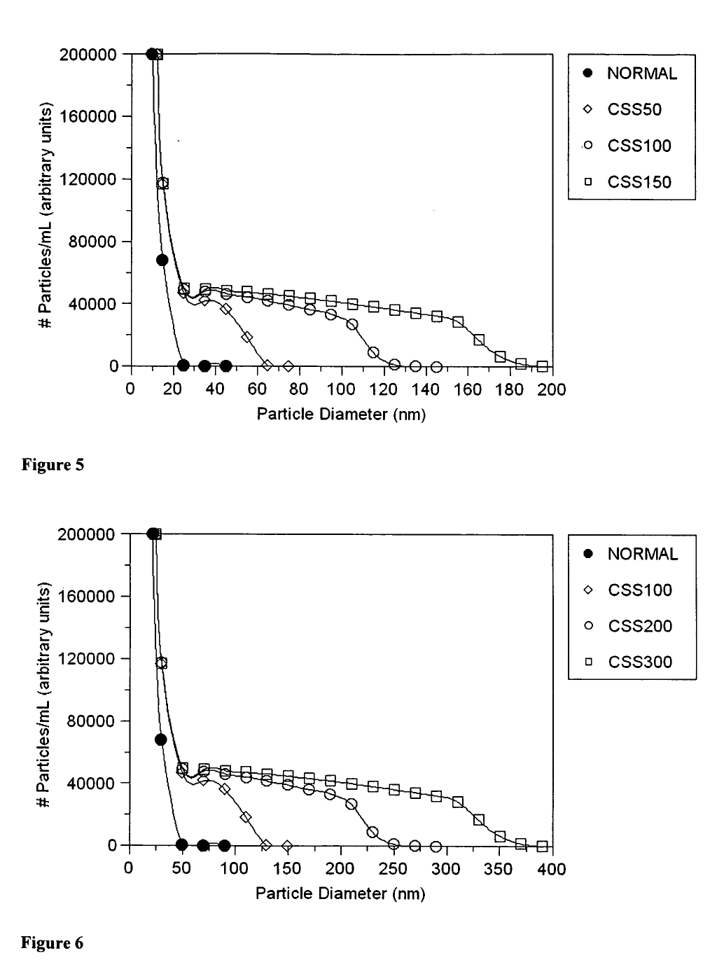

FIGS. 1-4 depict various hypothetical possibilities of “micro-size” structured dispersions as summarized in the top section of Table 1. Each of these figures includes a “fine” dispersion, referred to as “normal” (closed black circles), which arbitrarily was chosen to have a mean particle size (MPS) of 0.5 um. This serves as a useful reference for comparison to the three structured dispersions that are included in each figure. The PSDs for the structured dispersions were constructed by adding an appropriately broad distribution to the starting “normal” PSD and then “cutting off” the resulting large-size “tail” by use of an appropriate “filter function”. The amplitudes (concentrations) of the broad added PSDs were arbitrarily chosen to be one-tenth of the amplitude (concentration) of the “normal” dispersion—500,000 particles/mL at the MPS (peak) of 0.5 um. It must be emphasized that these particular values have been arbitrarily chosen, only for the purpose of creating these stylized plots. The resulting large-diameter tails with cut-off function yield specific, controlled “embolic” threshold values. In FIG. 1, these size thresholds are referred to as “EMB5”, “EMB 10” and “EMB 15”, corresponding to large-size thresholds (cutoffs) of 5, 10 and 15 um, respectively. In FIGS. 2, 3 and 4, the embolic threshold sizes shown are successively larger—10, 20 and 30 um (FIG. 2); 30, 60 and 90 um (FIG. 3); 60, 120 and 180 um (FIG. 4). The specific shapes and embolic thresholds of the large-diameter tails (LDTs) plotted in these figures are meant only to illustrate the concept of structured PSDs. The actual values chosen for the amplitudes, mean particle sizes and SD values of the functions that were used to generate the final structured PSDs are not meant to limit in any way the generality of the concept of structured PSDs discussed herein. In these examples, as with the nano-size dispersion examples discussed below, they illustrate a relative modification of the rapidly-decaying large-diameter tail that accompanies a normal, “fine” dispersion, but which extend out to much larger sizes, and which have additional purposes, than the LDTs that accompany “nano-size” structured dispersions, shown in FIGS. 5-8. In addition to controlling delivery, by virtue of their larger dimensions, these micro-size particle or droplet populations may offer one or more valuable mechanical functions, including deliberate deposition or occlusion/embolization within the microvasculature of the venous circulation (e.g., capillaries, arterioles, venules), or even within larger blood vessels, including the arterial circulation that supplies a vital organ system. The design of the particle size-structured dispersion will be dictated by clinical indications and/or the target site(s). Again, there are numerous permutations that are applicable, which would be known to those skilled in the art of formulation.

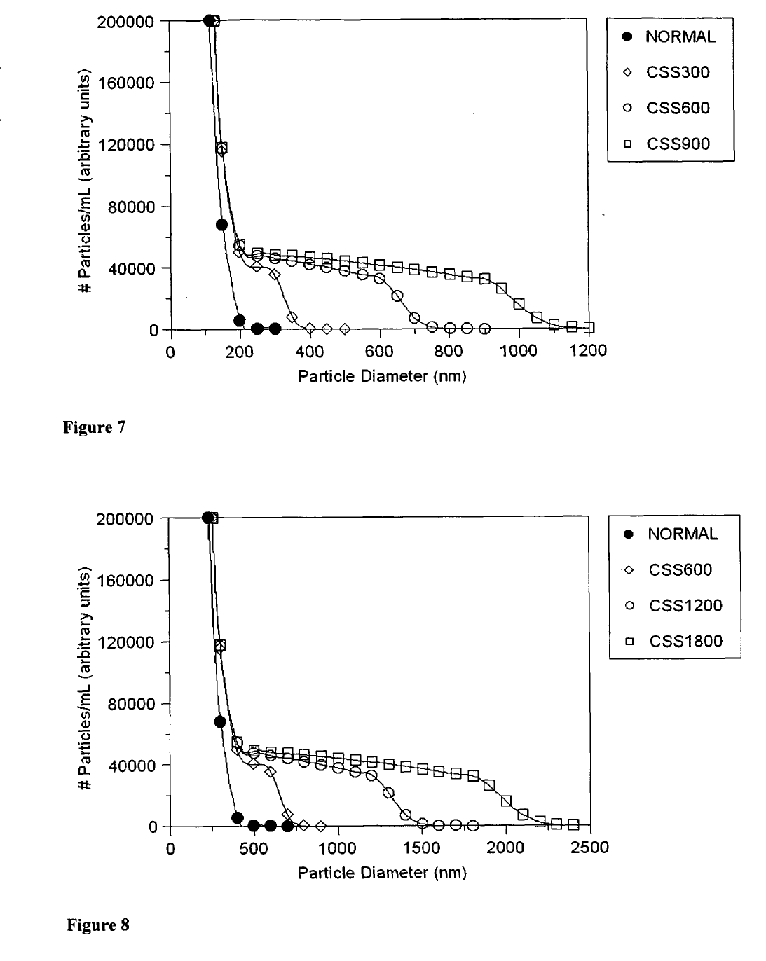

In contrast, FIGS. 5-8 depict examples of structured dispersions containing large-diameter tails that correspond to the hypothetical “nano-size” structured dispersions summarized in the lower section of Table 1. As in the case of the “micro-size” structured dispersions discussed above, the relatively rapidly-decaying LDT associated with a “normal”, “fine”, “unstructured”, “nano-size” dispersion is included in each figure (closed black circles) in order to provide a reference for the large-size, much-slower decaying tails of the other hypothetical, size-structured nano-dispersions. In FIG. 5 the “normal” PSD was arbitrarily chosen to have an MPS of 5 nm. In FIGS. 6, 7 and 8 the MPS values were arbitrarily chosen to be higher: MPS=10 nm (FIG. 6); MPS=50 nm (FIG. 7); MPS=100 nm (FIG. 8). The threshold, or “cut-off” size associated with each “nano-size” structured, extended tail is referred to as the characteristic structured size, or “CSS”. In FIG. 5, the size thresholds are therefore referred to as “CSS50”, “CSS100” and “CSS150”, corresponding to large-size thresholds (cutoffs) of 50, 100 and 150 nm, respectively. In FIGS. 6, 7 and 8, the characteristic structured size thresholds shown are successively larger—100, 200 and 300 nm (FIG. 6); 300, 600 and 900 nm (FIG. 7); 600, 1200 and 1800 um (FIG. 8). The differences in the size range, or extent, of the structured tails used in these examples of “nano-size” structured dispersions are again merely intended to provide an illustration of the diversity of possible applications. The size ranges embraced by the LDTs can clearly be modified as desired by the formulator in order to achieve a particular function or outcome, such as providing controlled delivery of a drug in the same or in different concentrations within a certain size range, as well as, for example, providing different doses of drugs for diseases, requiring the delivery of multiple drug regimens. Hence, the “terms” assigned to the data points would typically be chosen so to be reflective of the therapeutic intent. Therefore, instead of using the terms “EMB” or “DEP” (for “deposition”) or “CSS”, introduced above, one might one might usefully utilize, for example, the terms CR50, CR100 and CR150 in FIG. 5, in order to suggest the intended function of “controlled release” at the sizes indicated. The nano-size structured dispersions, by virtue of their principal role as a drug vehicle, would be commonly administered intravenously. It is, however, conceivable that some particle size-structured dispersions might contain a very wide size distribution of vesicles and droplets/globules (i.e., from nano- to micro-sized particles). In these cases, the dispersions may also be administered intra-arterially. There are clearly many other permutations of this concept that are applicable, which would be known to those skilled in the art of formulation.

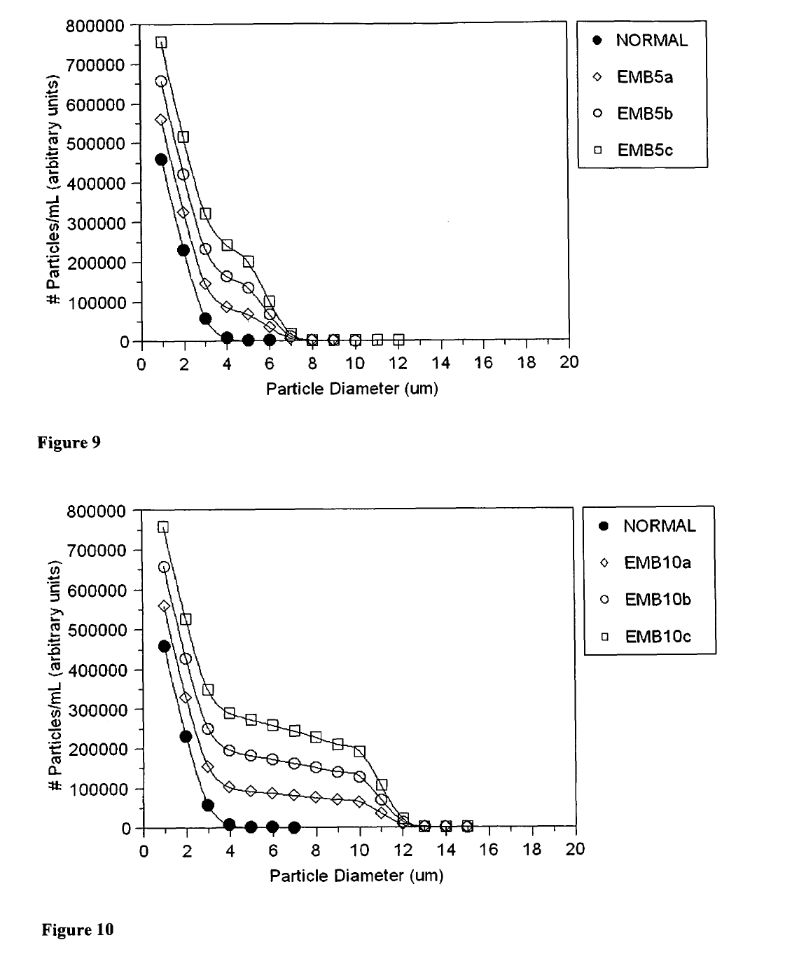

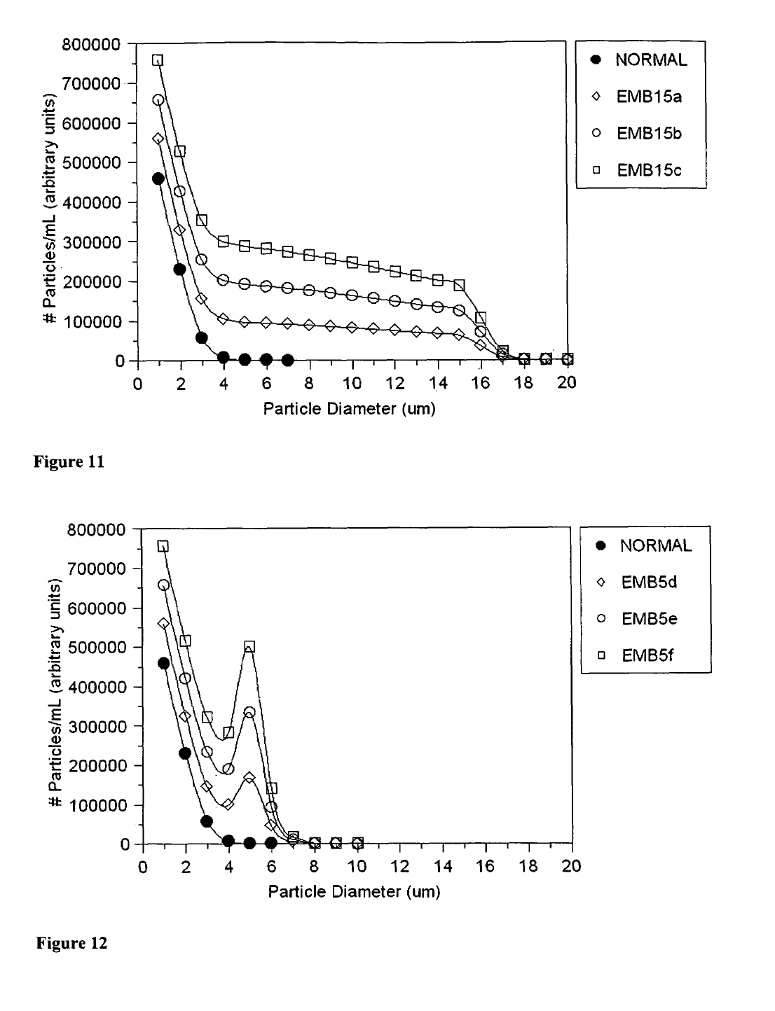

It may be desirable to broaden the characteristic size or concentration, or both, of the large-diameter tail portion of the structured PSD, including the addition of selected peak size(s) or diameter(s). This may be done in order to create a dispersion with progressively higher numbers of particles or droplets focused toward a certain single peak size, which may, for example, be done in order to achieve a pharmacological effect equivalent to administering a “loading dose” of a drug or adjuvant at the “target site”. Repeated doses at selected intervals, for example, may augment the therapeutic effect following the loading dose. FIGS. 9-11 show stylized plots of possible structured PSDs that are designed to achieve some of these ends, in which the controlled variable is the concentration of particles populating the large-size tail, for a given embolic threshold size. As before, each of these figures includes the “normal”, fast-decaying tail of a conventional (“normal”) “fine” dispersion (closed black circles) as a reference for comparison to three size-structured PSDs. The LDTs shown in FIG. 9 are all associated with the same characteristic, or “embolic” (“EMB”) threshold size—5 um. The three plots labeled EMB5a, EMB5b and EMB5c represent three increasing large-size particle concentrations (number/mL) for the same “threshold” (“EMB”) size. FIG. 10 shows a similar comparison of the LDTs associated with increasing large-size particle concentrations, but where the embolic threshold size is 10 um. FIG. 11 shows a similar comparison, but where the embolic threshold size is yet larger—15 um.

In the case of “chemoembolization”, a possible therapeutic approach involving a structured dispersion would be to provide successively higher amounts of a drug to the tumor with each incremental increase in the particle or droplet size. This point can be illustrated by starting with a conventional “normal”, “fine” dispersion having, for example, a mean droplet size of 0.5 μm, as utilized in FIG. 1. Structuring of this dispersion could then consist, for of adding, or superimposing, a relatively narrow “peak” of particles centered in size at 5 um, for example, onto the rapidly-decaying tail of the “normal” PSD. This structuring is depicted schematically in FIG. 12 for three different concentrations of particles populating the 5-um peak. For example, if 10 picograms (pg) of an active ingredient are contained in one 0.5-pm droplet, and the same proportion (mass/volume) of drug is maintained throughout the PSD of the peak-structured dispersion, then progressively higher concentrations of the active ingredient will be found with increasing droplet size, as depicted in Table 2. In this case, the amount of the active ingredient is constant across all populations, but in another case, the concentration of active ingredient may be designed to differ according to the selected structure of the particle or droplet/globule size and the particle concentration (number/mL), as shown in Table 3. For example, in the case of a dispersion intended to treat a lung infection, it may be desirable to construct a formulation with one globule population containing a high concentration of active ingredient(s) for those globules of sufficient size designed to lodge into the pulmonary circulation system, and another population containing much smaller droplets containing a small fraction of the drug concentration, designed to achieve systemic effects.

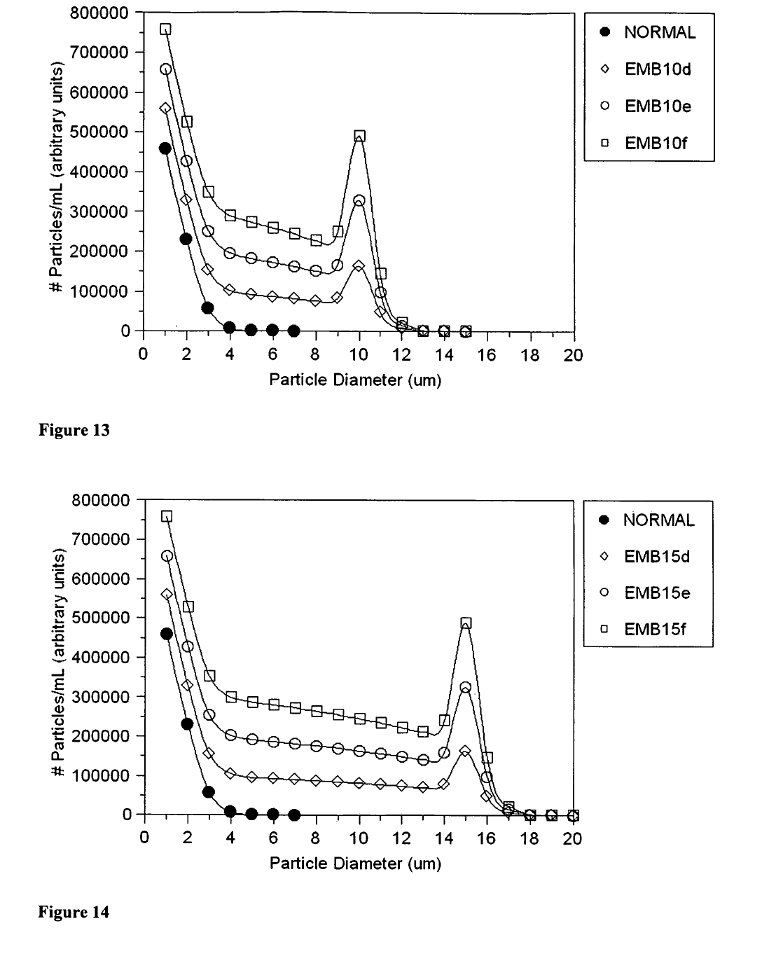

In another embodiment of the disclosure, it may be desirable to alter the large-diameter tail of the PSD in an effort to heighten more modestly the concentration of droplets/globules associated with one or more selected peaks that have been added to the PSD in the structuring process. This might be done to enhance the “lodging” of drug- and/or adjuvant-loaded particles or droplets/globules so that they can accumulate efficiently at the selected “target” site(s). Once “lodged”, the particles or droplets/globules could be designed to biodegrade slowly (compared to other droplets present), producing longer exposure or contact time between the active ingredient(s) and the target site(s). FIGS. 12-14 show stylized plots comparing the fast decaying tail of a “normal”, “fine” (unstructured) dispersion (closed black circles) against three LDTs of size-structured dispersions which now possess a single structured peak of successively increasing mean size—5 um (FIG. 12), 10 um (FIG. 13) and 15 um (FIG. 14)—in order to demonstrate an application of this concept. To further illustrate this example, and assuming 20 nanograms (ng) of an active ingredient are contained in each 5-μm droplet/globule utilized in FIG. 12, the final dose may be controlled based on the selection of various formulation factors such as: a) concentration of active ingredient(s); b) concentration of globules/mL; and c) size(s) of globules. The amount of active ingredient(s) can be expressed using various units. For example, units of mass can be expressed as grams, g; milligrams, mg (10⁻³ g); micrograms, μg (10⁻⁶ g); nanograms, ng (10⁻⁹ g); picograms, pg (10⁻¹² g); or femtograms, fg (10⁻¹⁵ g). Units of volume can be expressed as liters, L; milliliters, mL; or microliters, uL. As well, the concentration of active ingredients may be expressed as a ratio of weight per volume, w/v (e.g., g/L, mg %); weight per weight, w/w (e.g., g/g, pg/mg) and volume per volume, v/v (e.g., mL/mL, mL/L). From the various weight and volume relationships, additional terms may be used to express the amount of active ingredient(s), such as molarity, molality, normality and parts-per-million.

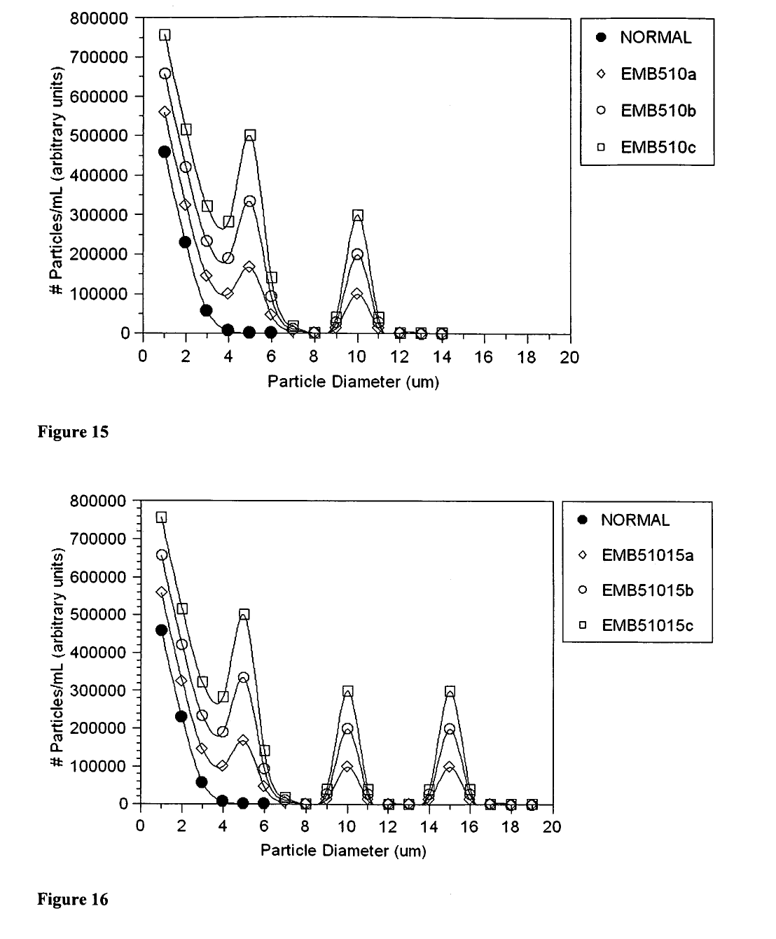

In another embodiment of the disclosure, it may also be desirable to further alter the PSD by structuring it not only by increasing the concentration of particles or droplets/globules associated with a single peak in size, but also by creating multiple peaks of two or more sizes with possibly different concentrations of both the active ingredient(s) and concentration of droplets/globules associated with each peak. This approach may offer various additional therapeutic possibilities. In one case, as in the previous example, the choice of a multiple-peak-structured PSD can facilitate the “lodging” of certain droplet/globule populations at the target site(s), thereby increasing the local concentration of the drug until it is appropriately biodegraded. Secondly, structuring the large-diameter tail of the PSD with additional “peaks” may increase the “contact time” of the drug at various locations in the surrounding vasculature that supplies the target site. This might include a population of large-diameter particles or droplets/globules that simultaneously occlude certain blood vessels, as with certain forms of “chemoembolization” therapy. FIGS. 15 and 16 show additional stylized plots, comparing the usual fast decaying tails associated with a normal, “fine” (unstructured) dispersion (solid black circles) against LDTs that are structured to contain two or more peaks of incrementally increasing size in order to possess the desired effect(s). FIG. 15 shows LDTs that contain two peaks, centered at 5 and 10 um, while FIG. 16 shows LDTs that contain three peaks, centered at 5, 10 and 15 um. It is also possible to produce individual particle size-structured dispersions, which can be administered separately via the same injection site or via another parenteral route in order to achieve multiple therapeutic effects.

In another embodiment of the disclosure, it may be desirable to alter the rate of biodegradation of the selected peak population(s) of the particle size-structured parenteral dispersion in order to achieve a desired effect (e.g., delivery of active ingredient(s), deposition, embolization, or any combination thereof). This may be accomplished, for example, by using a dispersion having a single peak in the PSD or GSD, so as to cause the particles or globules to temporarily “lodge” at the target site and “timed” so as to produce a high local concentration of active ingredient(s). Alternatively, a “single-peak” structured dispersion may be “timed” so as to “occlude” the vascular supply in an attempt, for example, either to induce ischemia and cell death, or to staunch blood flow to an aneurysm, equivalent to “chemical cauterization”. With these types of structured dispersions, a dosage regimen may be determined to optimize the response to the active ingredient(s). Similarly, in a structured dispersion having more than one peak in its PSD or GSD, each peak may possess its own biodegradation rate according to its intended effect. Thus, some structured dispersions may possess several characteristics that are designed to address the underlying disease condition in a number of ways, or by means of a number of different “mechanisms”.

In another embodiment of the disclosure, it may also be desirable to reduce the breadth, or width, of the range of sizes of the particles or droplets populated around the mean diameter or peak size(s) of the structured distribution, so that the dispersion is greatly narrowed—i.e., so that the population associated with each peak is nearly monodisperse, or uniform, in size. Minimizing or shortening the size range of the large-diameter tail of the PSD by whatever means appropriate may be beneficial by virtue of reducing unnecessary exposure of the host or patient to a population of overly large particles or globules that might be more than three standard deviations (SDs) larger than the mean size. Such “outlier” particles or globules are sometimes the source of heightened adverse events during an infusion. Therefore, reducing or eliminating altogether this population may be highly desirable and also potentially of great significance when the particles or droplets/globules are loaded with an active ingredient that possesses a narrow therapeutic index. The selected single peak can thus be formulated to be of any desired size in order to produce the desired local or systemic effects of the parenteral dispersion. As well, this capability may be particularly desirable in the case of drugs having a narrow therapeutic index in order to minimize toxicity associated with systemic exposure.

These examples are not meant to be inclusive, as there are numerous permutations and combinations of the ways that the peaks and/or large-diameter tail in the PSD of the size-structured dispersion may be manipulated in order to achieve a specific, optimal outcome. Rather, they are merely intended to emphasize the favorable possibilities that can occur using the methods and principles taught in this disclosure.

It should be implicitly understood from the teaching herein that in order to maintain the desired physical attributes of a given particle size-structured dispersion, the composition of a given dispersion may desirably be made to differ substantially from that of another dispersion, with each formulation containing a unique set of ingredients. Use of the term “composition”, referring to the final dispersion, is meant to include the components of both the aqueous and non-aqueous phases of the formulation. Whereas the aqueous phase primarily includes sterile water for injection, it may also contain pharmaceutical adjuvants or excipients, included to improve the quality, integrity and safety of the final product. Such components include, for example, buffers for pH control, antimicrobial agents and preservatives, emulsifying and/or co-emulsifying agents and tonicity enhancers. Similarly, the non-aqueous components include various concentrations and sizes of particles or droplets in the dispersed phase, which may be made from, for example, lipids or polymers. In addition, the non-aqueous phase may also include such components as active ingredients, emulsifying and/or co-emulsifying agents and antioxidants.

It is also implicitly understood from this disclosure that the method of producing the various desired particle size-structured dispersions will require specific preparation steps. These would include, for example, all aspects of homogenizations procedures necessary to yield the desired PSD for its intended purpose. These steps may be dependent on the unique composition of the formulation. Such achievements may be facilitated through the use of particle sizing techniques, such as those described above, as well as previously referenced techniques (Nicoli et al, 2006).

It should also be implicitly understood that specific combinations of total ingredients, including, for example, specific compositions and proportions of aqueous and non-aqueous components, can be derived from this disclosure, including development of the means for stabilization of the final dosage form. Thus, practice of the methods and concepts taught herein is expected to yield multiple new pharmaceutical entities and methods of pharmaceutical preparation, especially for those skilled in the art.

Tables mentioned herein are set forth below:

TABLE 1
Structured Large-Diameter Dispersions: Mean Particle Size (MPS)
and Relevant, Structured Large-Diameter Tail (LDT) Populations.

| | | | |
|---|---|---|---|
| MPS | LDTmin | LDTmax | Structured LDT Range |

Micrometer (μm) Ranges

| | | | | | | | |
|---|---|---|---|---|---|---|---|
| 0.5 | μm | 5 | μm | 25 | μm | 2-20 | μm |
| 1.0 | μm | 10 | μm | 50 | μm | 4-40 | μm |
| 5.0 | μm | 50 | μm | 250 | μm | 20-200 | μm |
| 10.0 | μm | 100 | μm | 500 | μm | 40-400 | μm |

Nanometer (nm) Ranges

| | | | | | | | |
|---|---|---|---|---|---|---|---|
| 5 | nm | 50 | nm | 250 | nm | 20-200 | nm |
| 10 | nm | 100 | nm | 500 | nm | 40-400 | nm |
| 50 | nm | 500 | nm | 2500 | nm | 200-2000 | nm |
| 100 | nm | 1000 | nm | 5000 | nm | 400-4000 | nm |

TABLE 2
A Dispersion Designed to Contain 10 picograms (pg) of Active
Ingredient per 0.5 μm Droplet Size Across a Range of Droplets*

| | Dose of Active |
|---|---|
| Droplet Size | Ingredient |
| 0.5 μm | 10 pg |
| 1.0 μm | 80 pg |
| 2.0 μm | 640 pg |
| 3.0 μm | 2,156 pg |
| 4.0 μm | 5,122 pg |
| 5.0 μm | 10,000 pg |

*Assumes dose is proportionate (mass/volume) across all droplet sizes.

TABLE 3
Dispersions Designed to Contain Multiple Concentrations of
Active Ingredient(s) per Globule Size Across 3 Peak Sizes.

| | Dose (“ng”) | | Dose (“mg”) |
|---|---|---|---|
| Globule Size | per Globule | Globules/mL | per mL |
| 5 μm | 20 ng | 225,000 | 4.5 mg |
| | | 450,000 | 9.0 mg |
| | | 675,000 | 13.5 mg |
| 10 μm | 500 ng | 75,000 | 37.5 mg |
| | | 150,000 | 75.0 mg |
| | | 22,500 | 11.25 mg |
| 15 μm | 40 ng | 150,000 | 6.0 mg |
| | | 300,000 | 12.0 mg |
| | | 450,000 | 18.0 mg |

All of the above-mentioned references are herein incorporated by reference in their entirety to the same extent as if each individual reference were specifically and individually indicated to be incorporated herein by reference in its entirety.

While various embodiments are described herein, it will be appreciated that variations, modifications and other changes in form and detail may be made without departing from the spirit and the scope of the disclosure. Such variations and modifications are to be considered within the purview and scope of the disclosure as defined by the appended claims.

1. A Drug/Adjuvant Delivery System (D/A DS) comprising:
a liquid carrier; and
a particle-size structured dispersion of solid and/or liquid particles suspended in the liquid carrier.

2. The D/A DS of claim 1, wherein said D/A DS is structured to treat a disease.

3. The D/A DS of claim 1, wherein said D/A DS is tailored to a specific D/A.

4. The D/A DS of claim 1, wherein said D/A DS is tailored to a specific disease.

5. The D/A DS of claim 1, wherein a particle size distribution (PSD) of said dispersion comprises a single, narrow peak center.

6. The D/A DS of claim 1, wherein the PSD of said dispersion comprises multiple, narrow peaks.

7. The D/A DS of claim 1, wherein the PSD of said dispersion comprises a single wide peak or center.

8. The D/A DS of claim 1, wherein the PSD of said dispersion comprises multiple, wide peaks.

9. The D/A DS of claim 1, wherein the PSD of said dispersion comprises a large-diameter tail with a single peak with a narrow width.

10. The D/A DS of claim 1, wherein the PSD of said dispersion comprises a large-diameter tail with a single peak with a wide width.

11. The D/A DS of claim 1, wherein the PSD of said dispersion comprises a large-diameter tail with a single peak, with a progressive rise to peak.

12. The D/A DS of claim 1, wherein the PSD of said dispersion comprises a large-diameter tail with multiple peaks with a narrow width.

13. The D/A DS of claim 1, wherein the PSD of said dispersion comprises a large-diameter tail with multiple peaks with a wide width.

14. The D/A DS of claim 1, wherein the PSD of said dispersion comprises a large-diameter tail with multiple peaks, with a progressive rise to each peak.

15. The D/A DS of claim 1, wherein the PSD of said dispersion comprises a large-diameter tail with multiple peaks, with a progressive rise to a selected peak(s).

16. The D/A DS of claim 1, wherein the PSD of said dispersion comprises a large-diameter tail that has been modified to deliver a D/A of a specified structure.

17. The D/A DS of claim 16, wherein the particles comprising the large-diameter tail are of a sufficient size to lodge at specific target site(s).

18. The D/A DS of claim 17, wherein the lodging produces highly localized delivery of D/A.

19. The D/A DS of claim 17, wherein the lodging produces a high concentration of D/A.

20. The D/A DS of claim 17, wherein the lodged D/A biodegrades in a desired manner.

21. The D/A DS of claim 20, wherein the biodegradation time can be controlled to selected time(s).

22. The D/A DS of claim 20, wherein the biodegradation time will determine a next dosing interval.

23. A method for chemoembolization or chemodeposition, comprising:
administering a clinically assessed effective amount of the D/A DS of claim 1 to a patient in need thereof.

24. The method of claim 23, wherein a response to the administration of the D/A is clinically assessed to determine additional course(s) of D/A DS.

25. The method of claim 23, wherein the response to the administration of the D/A is clinically assessed to determine an optimization of a dose of D/A DS.

26. The method of claim 23, wherein the response to the administration of the D/A is clinically assessed to determine an additional course of a modified D/A DS.

27. The method of claim 23, wherein the response to the administration of the D/A is clinically assessed to structure narrow vs. broad peaks.

28. The method of claim 23, wherein the response to the administration of the D/A is clinically assessed to structure single vs. multiple peaks.

29. The method of claim 23, wherein the response to the administration of the D/A is clinically assessed to determine narrow/broad and single/multiple peak combinations.

30. The method of claim 23, wherein the amount administered is effective to provide an embolization effect to occlude a blood supply.

31. The method of claim 30, wherein the particle size distribution (PSD) of said dispersion comprises a large-diameter tail with a single peak, with a progressive rise to peak.

32. The method of claim 30, wherein the PSD of said dispersion comprises a large-diameter tail with multiple peaks with a narrow width.

33. The method of claim 30, wherein the PSD of said dispersion comprises a large-diameter tail with multiple peaks with a wide width.

34. The method of claim 30, wherein the PSD of said dispersion comprises a large-diameter tail with multiple peaks, with a progressive rise to each peak.

35. The method of claim 30, wherein the PSD of said dispersion comprises a large-diameter tail with multiple peaks, with a progressive rise to a selected peak(s).

36. The method of claim 30, wherein the PSD of said dispersion comprises a large-diameter tail that has been modified to deliver D/A of a specified structure.

37. The method of claim 36, wherein the particles comprising the large-diameter tail are of a sufficient size to lodge at specific target site(s).

38. The method of claim 37, wherein the lodging produces highly localized delivery of D/A.

39. The method of claim 37, wherein the lodging produces a high concentration of D/A.

40. The method of claim 37, wherein the lodged D/A biodegrades in a desired manner.

41. The method of claim 40, wherein the biodegradation time can be controlled to selected time(s).

42. The method of claim 40, wherein the biodegradation time will determine a next dosing interval.

43. The method of claim 23, wherein the amount administered is effective to provide an embolization effect to occlude the blood supply and to provide pharmacological lodging.

44. The method of claim 43, wherein the PSD of said dispersion comprises a large-diameter tail with a single peak, with a progressive rise to peak.

45. The method of claim 43, wherein the PSD of said dispersion comprises a large-diameter tail with multiple peaks with a narrow width.

46. The method of claim 43, wherein the PSD of said dispersion comprises a large-diameter tail with multiple peaks with a wide width.

47. The method of claim 43, wherein the PSD of said dispersion comprises a large-diameter tail with multiple peaks, with a progressive rise to each peak.

48. The method of claim 43, wherein the PSD of said dispersion comprises a large-diameter tail with a multiple peaks, with a progressive rise to a selected peak(s).

49. The method of claim 43, wherein the PSD of said dispersion comprises a large-diameter tail that has been modified to deliver D/A.

50. The method of claim 49, wherein the particles comprising the large-diameter tail are of a sufficient size to lodge at specific target site(s).

51. The method of claim 50, wherein the lodging produces highly localized delivery of D/A.

52. The method of claim 50, wherein the lodging produces a high concentration of D/A.

53. The method of claim 50, wherein the lodged D/A biodegrades in a desired manner.

54. The method of claim 53, wherein the biodegradation time can be controlled to selected time(s).

55. The method of claim 53, wherein the biodegradation time will determine a next dosing interval.

56. The D/A DS of claim 1, wherein a dose of D/A is the same for all droplets.

57. The D/A DS of claim 1, wherein a dose of D/A is “peak” size-specific.

58. The D/A DS of claim 1, wherein a dose of D/A is globule concentration-specific.

59. The D/A DS of claim 1, wherein a dose of D/A is PSD width-specific.

60. The D/A DS of claim 1, wherein a dose of D/A includes any combination of peak” size-specific, globule concentration-specific, and PSD width-specific.

61. The D/A DS of claim 1, wherein droplets or globules of the particle-size structured dispersion are of a specified composition configured to biodegrade at similar rates.

62. The D/A DS of claim 1, wherein droplets or globules of the particle-size structured-dispersion are of a specified composition configured to biodegrade at different rates.

63. The D/A DS of claim 1, wherein the PSD of the dispersion comprises one peak.

64. The D/A DS of claim 1, wherein the PSD of the dispersion comprises multiple peaks.

65. The D/A DS of claim 64, wherein each of the multiple peaks has the same width.

66. The D/A DS of claim 64, wherein the multiple peaks have multiple widths.

67. The D/A DS of claim 1, wherein droplets or globules of the PDS are configured to biodegrade.

68. The D/A DS of claim 1, wherein the D/A DS dispersion is relatively low in toxicity.

69. The D/A DS of claim 1, wherein the D/A DS dispersion provides a relatively high total dose(s).

70. The D/A DS of claim 1, wherein biodegradability is controlled for each peak in the large-diameter tail of the structured dispersion so as to allow sequenced or chronological breakdown of infused globules.

71. The D/A DS of claim 70, wherein the biodegradability possesses information relating to its transition time(s) to its biodegradation to identify optimal dose regimen.

72. The D/A DS of claim 70, wherein the biodegradability maintains a desired local concentration of active ingredient.

73. The D/A DS of claim 70, wherein the biodegradability allows tailoring of the structured dispersion to a target site.

74. The D/A DS of claim 1, wherein the D/A DS is a single-purpose, single-mechanism dispersion.

75. The D/A DS of claim 1, wherein the D/A DS is a single-purpose, single-composition, single-mechanism dispersion.

76. The D/A DS of claim 1, wherein the D/A DS is a single-purpose, single composition, multiple-mechanism dispersion.

77. The D/A DS of claim 1, wherein the D/A DS is a multiple-purpose, single-mechanism dispersion.

78. The D/A DS of claim 1, wherein the D/A DS is a multiple-purpose, single-composition, single-mechanism dispersion.

79. The D/A DS of claim 1, wherein the D/A DS is a multiple-purpose, multiple-composition, single-mechanism dispersion.

80. The D/A DS of claim 1, wherein the D/A DS is a multiple-composition, multiple-mechanism, multiple-mechanism dispersion.

81. The D/A DS of claim 1, wherein the particle-size structured dispersion comprises a peak structured to include particles having a specified mean particle size of at least about 1 micrometer.

82. The D/A DS of claim 81, wherein the dispersion is a stable dispersion.

83. The D/A DS of claim 82, wherein an interparticle potential energy barrier inhibits neighboring particles from approaching each other closely enough to permit irreversible agglomeration due to short-range attractive forces.

84. The D/A DS of claim 81, wherein the particles comprising the peak have a mean particle size of about 5 micrometers or more.

85. The D/A DS of claim 81, wherein the peak comprises about 1% or more of particles of an overall dispersed phase on a mass- or volume-weighted basis.

86. The D/A DS of claim 81, wherein the particles comprising the peak have a size distribution wherein the size range of the distribution is less than about 20% of the mean particle size of the particles comprising the large diameter tail.

87. The D/A DS of claim 81, wherein the overall mean particle size of the particles in the dispersion is at least ½ to 1 log below the mean particle size of the particles in peak.

88. The D/A DS of claim 81, wherein the PSD comprises a unimodal large-diameter tail comprising a single peak.

89. The D/A DS of claim 81, wherein the PSD comprises a multimodal large-diameter tail comprising multiple peaks.

90. The D/A DS of claim 81, wherein the liquid carrier is aqueous.

91. The D/A DS of claim 81, wherein the particles comprise one or more lipids and/or polymers.

92. The D/A DS of claim 81, wherein the particles comprise one or more active agents.

93. The D/A DS of claim 81, wherein the dispersion comprises one or more pharmaceutically acceptable excipients.

94. The D/A DS of claim 81, wherein the particle size distribution of the dispersion is determined using a single-particle optical sizing technique.

95. The D/A DS of claim 81, wherein the particles are biodegradable.

96. The D/A DS of claim 81, wherein the particles are non-biodegradable.

97. A method for embolization, comprising administering an effective amount of the dispersion of claim 81 to a patient in need thereof.

98. The method of claim 97, wherein said dispersion is administered intra-arterially.

99. A method for chemodeposition, comprising administering an effective amount of the dispersion of claim 81 to a patient in need thereof.

100. The method of claim 99, wherein said dispersion is administered intravenously.

101. A particle-size structured dispersion comprising:
a liquid carrier; and
solid or liquid particles suspended in the liquid carrier, wherein a the particle size distribution (PSD) of said dispersion comprises a large diameter tail having a large diameter tail having a first peak of particles with a mean particle size of about 5 to about 10 micrometers and a second peak of particles having a mean particle size of about 15 to about 25 micrometers.

102. The dispersion of claim 101, wherein the particles of the first peak have a size distribution wherein a size range of the size distribution is less than about 20% of the mean particle size of particles comprising the first peak, and wherein the particles comprising the second peak have a size distribution wherein a size range of the distribution is less than about 20% of the mean particle size of the particles comprising the second peak.
